(19) United States
(12) Patent Application Publication
(10) Pub. No.: US 2013/0261180 A1
(43) Pub. Date: Oct. 3, 2013

(54) CLINICAL BENEFITS OF EICOSAPENTAENOIC ACID IN HUMANS

(71) Applicant: E I DU PONT DE NEMOURS AND COMPANY, Wilmington (DE)

(72) Inventors: Peter John Gillies, Pennington, NJ (US); Ernst John Schaefer, Natick, MA (US)

(73) Assignee: E I DU PONT DE NEMOURS AND COMPANY, Wilmington (DE)

(21) Appl. No.: 13/901,687

(22) Filed: May 24, 2013

Related U.S. Application Data
(63) Continuation of application No. 12/987,303, filed on Jan. 10, 2011.
(60) Provisional application No. 61/295,347, filed on Jan. 15, 2010.

(51) Int. Cl. A61K 31/202; A61K 31/232

(52) U.S. Cl. CPC A61K 31/202; A61K 31/232
USPC 514/547; 514/560

(57) ABSTRACT

Methods are provided for maintaining or lowering lipoprotein-associated phospholipase A₂ [“Lp-PLA₂”] levels, stabilizing rupture prone-atherosclerotic lesions, decreasing the Inflammatory Index and increasing Total Omega-3 Score™ in humans, by administering an effective amount of eicosapentaenoic acid [“EPA”], an omega-3 polyunsaturated fatty acid [“PUFA”].

This application claims the benefit of U.S. Provisional Application No. 61/295,347, filed Jan. 15, 2010 and which is hereby incorporated by reference in its entirety.

### FIELD OF THE INVENTION

This invention is in the field of biotechnology. More specifically, this invention pertains to methods of maintaining or lowering lipoprotein-associated phospholipase A₂ [“Lp-PLA₂”] levels, stabilizing rupture prone-atherosclerotic lesions, decreasing the Inflammatory Index and increasing Total Omega-3 Score™ in humans, by administration of eicosapentaenoic acid [“EPA”], an omega-3 polyunsaturated fatty acid [“PUFA”].

### BACKGROUND OF THE INVENTION

Health benefits derived from supplementation of the diet with omega-3 fatty acids, such as alpha-linolenic acid [“ALA”] (18:3), stearidonic acid [“STA”] (18;4), eicosatetraenoic acid [“ETrA”] (20:3), eicosatrienoic acid [“ETA”] (20;4), eicosapentaenoic acid [“EPA”] (20:5), docosapentaenoic acid [“DPA”] (22:5) and docosahexaenoic acid [“DHA”] (22:6), are well recognized and supported by numerous clinical studies and other published public and patent literature. For example, omega-3 fatty acids have been found to have beneficial effects on the risk factors for cardiovascular diseases, especially mild hypertension, hypertriglyceridemia and on coagulation factor VII phospholipid complex activity.

Despite abundant research in the area of omega-3 fatty acids, however, many past studies have failed to recognize that individual long-chain omega-3 fatty acids (e.g., EPA and DHA) are metabolically and functionally distinct from one another, and thus each may have specific physiological functions and biological activities.

This lack of mechanistic clarity is largely a consequence of the use of fish oils which contain a variable mixture of omega-3 fatty acids, as opposed to using pure EPA or pure DHA in clinical studies [the fatty acid composition of oils from menhaden, cod liver, sardines and anchovies, for example, comprise oils having a ratio of EPA:DHA of approximately 0.9:1 to 1.6:1 (based on data within The Lipid Handbook, 2^{nd} ed.; F. D. Gunstone, J. L. Harwood and F. B. Padley, Eds; Chapman and Hall, 1994)].

There is a pharmaceutical composition sold under the trademark OMACOR® and now known as LOVAZA™ [U.S. Pat. No. 5,502,077, No. 5,656,667 and No. 5,698,594] (Pronova Biocare A. S., Lysaker, Norway), that is a combination of ethyl esters of DHA and EPA. Each capsule contains approximately 430 mg/g-495 mg/g EPA and 347 mg/g-403 mg/g DHA with 90% (w/w) [“weight by weight”] total omega-3 fatty acids.

Intl. App. Pub. No. WO 2008/088415, published on 24 Jul. 2008, describes reducing lipoprotein-associated phospholipase A₂ [“Lp-PLA₂”] levels in patients, with primary hypertriglyceridemia or hypercholesterolemia or mixed dyslipidemia, coronary heart disease, vascular disease, atherosclerotic disease and vascular events in patients at risk thereof, by using omega-3 fatty acids, either as monotherapy or as combination therapy with a dyslipidemic agent. Use of pure EPA or pure DHA, as well as blended compositions having EPA:DHA ratios from 99:1 to 1:99, in treating such patients was mentioned; in preferred embodiments the EPA:DHA ratio is between 2:1 to 1:2. A randomized, double-blind, placebo-controlled clinical study was described in WO 2008/088415, performed to assess the efficacy and safety of combined LOVAZA™ and simvastatin therapy in hypertriglyceridemic subjects.

U.S. Pat. No. 7,498,359 issued Mar. 3, 2009 to Yokoyama et al., (Mochida Pharmaceutical, Ltd.) describes administration a high purity EPA ethyl ester [sold under the trademark Epadel® and Epadel® S in Japan] that is useful for reducing recurrence of stroke when administered in combination with a 3-hydroxy-3-methylglutaryl coenzyme A [“HMG-CoA”] reductase inhibitor.

WO 2010/093634 A1 published on Aug. 19, 2010 describes the use of EPA ethyl ester for treating hypertriglyceridemia.

Beebe et al., J. Chromatography 459:369-378 (1988), described preparative scale HPLC of omega-3 polyunsaturated fatty acid esters derived from fish oil.

GB Patent Application No. 1,604,554, published on Dec. 9, 1981 describes the use of EPa in treating thrombo-embolic conditions where in at least 50% by weight of the fatty acid composition should be EPA.

Satoh et al., Diabetes Care, 30(1):144-146 (January, 2007) examined the effects of purified EPA ethyl ester on atherogenic small dense LDL (sdLSL) particles, remnant lipoprotein particles, and C-reactive protein in metabolic syndrome.

Few studies have been performed with substantially pure EPA and separately with substantially pure DHA, to enable differentiation of the pharmacological effects of each individual fatty acid. One exception is the Japanese EPA Lipid Intervention Study [“JELIS”], which involved a large-scale randomized controlled trial using >98% purified EPA-ethyl esters (Mochida Pharmaceutical) in combination with a statin (Yokoyama, M. and H. Origasa, Amer. Heart J., 146:613-620 (2003); Yokoyama, M. et al., Lancet, 369:1090-1098 (2007)). It was found that cardiovascular events in patients receiving EPA plus statin decreased by 19% with respect to those patients receiving statin alone. This provides strong support that EPA, per se, is cardioprotective; similar studies using DHA have not been reported.

Notwithstanding the foregoing, the JELIS study did report changes in the serum ratio of arachidonic acid [“ARA”] (20:4, omega-6) to EPA. The JELIS study did not link these changes to Lp-PLA₂ or the Omega-3 Score™. Furthermore, the JELIS study did not consider the possible benefits of a relatively pure EPA as monotherapy (i.e., without coadministration of a statin), in either its natural triglyceride formor in an ethyl-ester form. From a biological perspective, EPA delivered as a triglyceride enters the blood circulation directly via the thoracic duct whereas EPA delivered as an ethyl-ester enters the blood after being shunted to the liver via the portal vein where it is subject to hepatic metabolism.

Omega-3 fatty acids at high doses are known to have significant triglyceride lowering properties. Four capsules per day of a concentrated formulation of omega-3 ethyl esters has been approved in the United States by the Food and Drug Administration for triglyceride lowering in patients with fasting triglycerides over 500 mg/dl. Each of these one gram capsules contains 465 mg of EPA and 375 mg of DHA, for a total dose of 1,860 mg of EPA and 1,500 mg of DHA in the 4 capsules. This formulation at this dose has been reported to decrease triglyceride levels by 29.5% and raise high-density lipoprotein [“HDL”] cholesterol by 3.4% versus placebo (both p<0.05) in subjects with triglyceride levels between 200 and 500 mg/dl on simvastatin 40 mg/day (Davidson, M. H. et al., Clin. Ther., 29:1354-1367 (2007). Even greater triglyceride reductions are observed in subjects with triglyceride levels over 500 mg/dl. It has been documented that this formulation lowers very low density lipoprotein apoB-100 levels by decreasing synthesis rates (Chan, D. C. et al., Am. J. Clin. Nutr., 77:300-307 (2003)). It has also been documented that DHA at doses of approximately 1200 mg/day will significantly lower triglyceride levels by about 25% (Davidson, M. H. et al., J. Am. Coll. Nutr., 16(3):236-243 (1997); Berson, E. L. et al., Arch. Opthalmol., 122:1297-1305 (2004)). In contrast, in the large JELIS trial, 1800 mg/day of EPA had no significant effect on triglyceride lowering.

Mori and colleagues have studied purified DHA and EPA each given at 4 grams/day versus olive oil placebo and documented that only DHA significantly increased forearm blood flow in response to acetylcholine infusion relative to placebo (Mori, T. A., et al., Circulation, 102:1264-1269 (2000)). In addition they showed that at these doses EPA reduced triglyceride levels by 18%, while DHA lowered these levels by 20% in overweight, hyperlipidemic men (Mori, T. A., et al., Am. J. Clin. Nutr., 71:1085-1094 (2000)). DHA also significantly increased HDL₂ cholesterol (Mori, T. A. et al., Am. J. Clin. Nutr., supra). The overall data suggest that at least 1200 mg/day of DHA is required for triglyceride lowering, while much higher doses of EPA are needed for this effect to be observed.

Omega-3 fatty acids, especially EPA, have been suggested to suppress the immune response. Mori and colleagues documented that 4 grams of either purified DHA or EPA per day had no significant effects on C-reactive protein [“CRP”], interleukin-6 [“IL-6”], or tumor necrosis alpha (Mori, T. A. et al., Free Radical Biology and Medicine, 35:772-781 (2003)). Phillipson, B. E. et al. (N. Engl. J. Med., 312:1210-1216 (1985)) have documented that very high doses of omega-3 fatty acids (i.e., one gram fish oil capsules/day) will suppress interleukin 1 and tumor necrosis factor alpha. Similarly, Meydani, S, N. et al. (J. Clin. Invest., 92:105-113 (1993)) have also shown that diets high in oily fish containing about 1200 mg/day of EPA and DHA will significantly reduce cell mediated immunity. In these studies by Meydani and colleagues, high fish diets decreased the percentage of helper T cells and increased the percentage of suppressor T cells, and significantly reduced the mitogenic response of mononuclear cells to concanavalin A and delayed type hypersensitivity skin responses, as well as the production of cytokines interleukin-1 [“IL-1”] beta, tumor necrosis factors [“TNF”], and IL-6 by mononuclear cells. Diets enriched in omega-6 polyunsaturated fats had the opposite effects as compared to an average American diet (Meydani et al., supra). These data indicate that EPA and DHA together as part of a high fish diet can suppress cell mediated inflammatory responses.

Most recently, Tull and colleagues elucidated a new step in neutrophil recruitment allowing for their passage across the endothelial layer (Tull, S. P. et al., PLoS. Biol., 7:e1000177 (2009)). The signal for this step is supplied when arachidonic acid [“ARA”] (20:4 omega-6) is metabolized into prostaglandin D2 by cyclooxygenase enzymes. If instead EPA is utilized and prostaglandin D3 is formed, there is an inhibition of neutrophil migration, and this may be why omega-3 fatty acids are protective of coronary heart disease development. Allayee, H. and colleagues (J. Nutrigenet. Nutrigenomics, 2:140-148 (2009)) have recently reviewed the implications that this type of inhibition may have for cardiovascular disease protection as it relates to the 5-lipoxygenase/leukotriene biosynthesis pathway. Bouwens, M. et al. (Am. J. Clin. Nutr., 90:415-424 (2009)) have documented that the combined daily intake of 1800 mg of EPA plus DHA resulted in significant reductions in the expressions of genes in mononuclear cells involved in inflammation and atherosclerosis such as nuclear transcription factor kappaB signaling, eicosanoid synthesis, scavenger receptor activity, adipogenesis, and hypoxia signaling.

Heretofore, the relative importance of EPA versus DHA has been unknown. Not only did human clinical trials discussed herein demonstrate the safety and efficacy of EPA-enriched oils, these trials also demonstrated that EPA and DHA have different biological effects. Specifically, the human clinical trials discussed herein demonstrate some surprising and unexpected nutritional and therapeutic benefits of EPA.

### SUMMARY OF THE INVENTION

In a first embodiment, the invention concerns a method for maintaining or lowering Lp-PLA₂ levels in a normal subject which comprises administering an effective amount of EPA. The initial Lp-PLA₂ level can be in the normal or borderline high range.

In a second embodiment, EPA can be in a triglyceride form in an oil that is low in saturated fatty acids.

In a third embodiment, the invention concerns a method for stabilizing a rupture prone-atherosclerotic lesion in a normal subject having a low level of serum EPA which comprises administering an effective amount of EPA. Furthermore, the subject can have a normal level of triglycerides or a high level of LDL or both.

In a fourth embodiment, the invention concerns a method for decreasing the Inflammatory Index in a normal subject which comprises administering an effective amount of EPA.

In a fifth embodiment, the invention concerns a method for increasing Total Omega-3 Score™ in a normal subject having a low level of serum EPA which comprises administering an effective amount of EPA.

In a sixth embodiment, the invention concerns a method for maintaining or lowering Lp-PLA₂ levels without raising LDL cholesterol levels in a normal subject which comprises administering an effective amount of EPA.

In a seventh embodiment, the invention concerns a method for maintaining or lowering Lp-PLA₂ levels without raising LDL cholesterol levels in a normal subject which comprises administering an effective amount of EPA wherein said method is for pre-emptive intervention in maintaining or lowering Lp-PLA₂ levels without raising LDL cholesterol levels in a normal subject having a low serum level of EPA.

In an eighth embodiment, the invention concerns using an effective amount of EPA that is substantially free of DHA in any of the methods disclosed herein.

In a ninth embodiment, the invention concerns a method for maintaining or lowering Lp-PLA₂ levels in a subject which comprises administering an effective amount of EPA substantially free of DHA. The initial Lp-PLA₂ level can be in the normal or borderline high range. Preferably, the EPA is in a triglyceride form in an oil that is low in saturated fatty acids.

In a tenth embodiment, the invention concerns a method for stabilizing a rupture prone-atherosclerotic lesion in a subject having a low level of serum EPA which comprises administering an effective amount of EPA substantially free of DHA. Preferably, with respect to this tenth embodiment, the subject has a normal level of triglycerides. Alternatively, or additionally, the subject may have a high level of LDL.

In an eleventh embodiment, the invention concerns a method for decreasing the Inflammatory Index in a subject which comprises administering an effective amount of EPA substantially free of DHA.

In a twelfth embodiment, the invention concerns a method for increasing Total Omega-3 Score™ in a subject having a low level of serum EPA which comprises administering an effective amount of EPA substantially free of DHA.

In a thirteenth embodiment, the invention concerns a method for maintaining or lowering Lp-PLA₂ levels without raising LDL cholesterol levels in a subject which comprises administering an effective amount of EPA substantially free of DHA.

In a fourteenth embodiment, the invention concerns a method for pre-emptive intervention in maintaining or lowering Lp-PLA₂ levels without raising LDL cholesterol levels in a subject having a low serum level of EPA which comprises administering an effective amount of EPA substantially free of DHA.

In a fifteenth embodiment, the invention concerns a method for lowering small dense LDL cholesterol (sdLDL) levels in a subject which comprises administering an effective amount of EPA substantially free of DHA.

In a sixteenth embodiment, the invention concerns a method for lowering small dense LDL cholesterol (sdLDL) levels in a normal subject which comprises administering an effective amount of EPA.

In a seventeenth embodiment, the invention concerns a method for stabilizing a rupture prone-atherosclerotic lesion in a subject having a low level of serum EPA which comprises administering an effective amount of EPA substantially free of DHA, in combination with an Lp-PLA₂ inhibitor wherein the Lp-PLA₂ inhibitor can be selected from the group consisting of as darapladib or rilapladib or a derivative of either.

### BRIEF DESCRIPTION OF THE DRAWINGS

FIG. 1 shows the effect of clinical treatments on serum EPA levels, while

FIG. 2 shows the effect of clinical treatments on serum DHA levels. Notably, EPA substantially free of DHA significantly raised the serum level of EPA in a dose-dependent manner.

FIG. 3 shows the effect of clinical treatments on the Inflammation Index. Notably, EPA substantially free of DHA significantly decreased the serum ratio of ARA/EPA in a dose-dependent manner.

FIG. 4 shows the effect of clinical treatments on the Total Omega-3 Score™. Notably, both EPA substantially free of DHA and DHA-enriched oils increased the Total Omega-3 Score™.

FIG. 5 shows the effect of clinical treatments on LDL cholesterol levels. Notably, EPA substantially free of DHA did not increase LDL cholesterol levels.

FIG. 6 shows the effect of clinical treatments on Lp-PLA₂ levels.

FIG. 7 is a regression analysis of EPA (substantially free of DHA)-enriched oils and DHA-enriched oils on Lp-PLA₂ levels. Results demonstrate that EPA has a statistically significant effect on Lp-PLA₂ levels, but DHA does not have such an effect.

### DETAILED DESCRIPTION OF THE INVENTION

All patent and non-patent literature cited herein are hereby incorporated by reference in their entirety.

In this disclosure, a number of terms and abbreviations are used. The following definitions are provided.

“American Type Culture Collection” is abbreviated as “ATCC”.

“Polyunsaturated fatty acid(s)” is abbreviated as “PUFA(s)”.

“Eicosapentaenoic acid” is abbreviated as “EPA”.

“Docosahexaenoic acid” is abbreviated as “DHA”.

“Triacylglycerols” are abbreviated as “TAGs”.

“Total fatty acids” are abbreviated as “TFAs”.

“Fatty acid methyl esters” are abbreviated as “FAMEs”.

“Dry cell weight” is abbreviated as “DCW”.

As used herein the term “invention” or “present invention” is intended to refer to all aspects and embodiments of the invention as described in the claims and specification herein and should not be read so as to be limited to any particular embodiment or aspect.

The term “fatty acids” refers to long chain aliphatic acids (alkanoic acids) of varying chain lengths, from about C₁₂ to C₂₂, although both longer and shorter chain-length acids are known. The predominant chain lengths are between C₁₆ and C₂₂. The structure of a fatty acid is represented by a simple notation system of “X:Y”, where X is the total number of carbon [“C”] atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” [“PUFAs”], and “omega-6 fatty acids” [“ω-6” or “n-6”] versus “omega-3 fatty acids” [“ω-3” or “n-3”] are provided in U.S. Pat. No. 7,238,482, which is hereby incorporated herein by reference.

“Eicosapentaenoic acid” [“EPA”] is the common name for cis-5,8,11,14,17-eicosapentaenoic acid. This fatty acid is a 20:5 omega-3 fatty acid. The term EPA as used in the present disclosure will refer to the acid or derivatives of the acid (e.g., glycerides, esters, phospholipids, amides, lactones, salts or the like) unless specifically mentioned otherwise.

“Docosahexaenoic acid” [“DHA”] is the common name for cis-4,7,10,13,16,19-docosahexaenoic acid. This fatty acid is a 22:6 omega-3 fatty acid. The term DHA as used in the present disclosure will refer to the acid or derivatives of the acid (e.g., glycerides, esters, phospholipids, amides, lactones, salts or the like) unless specifically mentioned otherwise.

“Triglycerides” [“TGs”] refer to the natural molecular form of lipids, wherein three fatty acids (e.g., EPA) are linked to a molecule of glycerol. Free fatty acids are rapidly oxidized and therefore the glycerol backbone helps to stabilize the EPA molecule for storage or during transport versus breakdown and oxidation. In contrast, “ethyl esters” [“EEs”] refer to a chemical form of lipids that are synthetically derived by reacting free fatty acids with ethanol. For example, this can occur during trans-esterification processing of some fish oils to produce “omega-3 fish oil concentrates”, as the fatty acids are cleaved from their natural glycerol backbone and then esterified, or linked, with a molecule of ethanol. Following trans-esterification, the ethyl esters typically undergo molecular distillation or short path evaporation. Ethyl ester fish oils could more appropriately be referred to as “semi-synthetic”, as both ethanol and fatty acids are natural—despite the fact that esterification of these two substances is not found in natural food sources of omega-3 fatty acids.

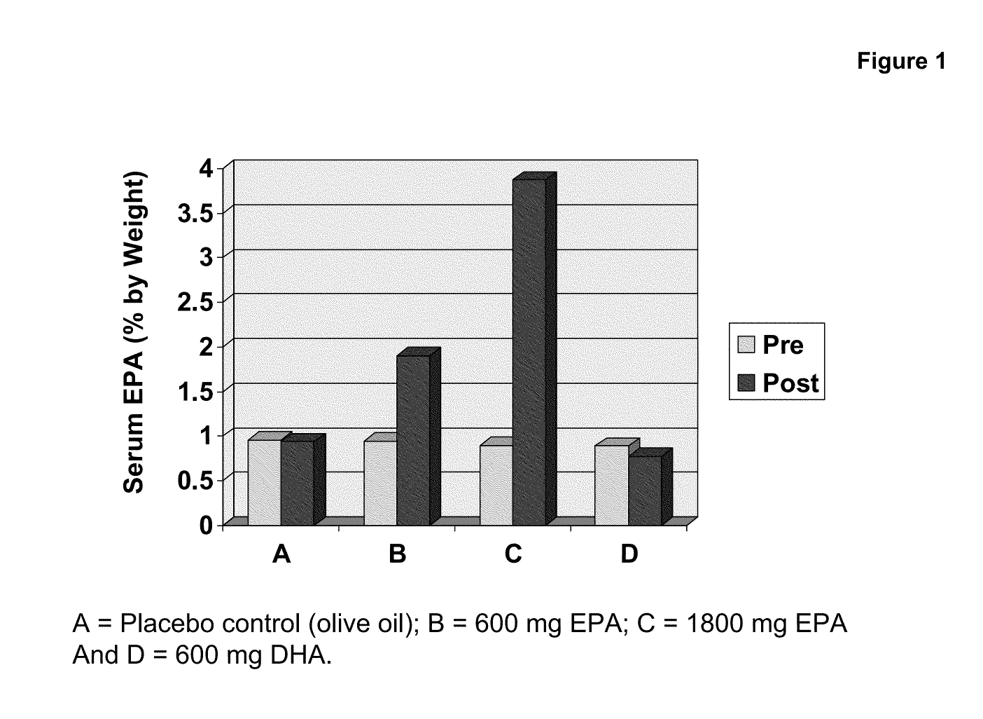

The term “an effective amount of EPA” refers to an amount of EPA sufficient to achieve the intended effects set forth herein. Preferably the “effective amount of EPA” is at least about 500 mg/day of EPA. More preferably, the “effective amount of EPA” is at least about 600 mg/day, this amount is based on the data set forth herein and in FIG. 1 attached hereto. Even more preferably, an effective amount of EPA is at least about 1200 mg/day and most preferably at least about 1800 mg/day. Although preferred dosages are described above, useful examples of dosages include any integer percentage between 500-1800 mg/day, although these values should not be construed as a limitation herein.

The percent of EPA with respect to the total fatty acids and their derivatives will be at least 10% or greater, while more preferably the composition is at least 20 EPA % TFAs, more preferably at least 30 EPA % TFAs, more preferably at least 40 EPA % TFAs, more preferably at least 50 EPA % TFAs, more preferably 60 EPA % TFAs, more preferably 70 EPA % TFAs, more preferably 80 EPA % TFAs, more preferably 90 EPA % TFAs and most preferably 95 EPA % TFAs. Any integer percentage between 10-100 EPA % TFAs will also be effective, although not specifically notated herein.

In some embodiments, it is contemplated that other omega-3 PUFAs may also be present in the EPA composition, such as DPA and DHA. If DHA is present in the composition, it is provided that the amount of DHA does not interfere with achieving the intended effects of EPA as set herein.

Preferably, the effective amount of EPA is substantially free of DHA, wherein “substantially free of DHA” means less than about 5.0 DHA % TFAs, more preferably less than about 1.0 DHA % TFAs, more preferably less than about 0.5 DHA % TFAs, or even most preferably less than about 0.1 DHA % TFAs, wherein the concentration of DHA within the total fatty acids is relative to the total oil. When the “effective amount of EPA” is “substantially free of DHA”, then a dosage of less than 600 mg/day may be possible, about less than 500 mg/day, provided that the amount of EPA is sufficient to achieve the intended effects set forth herein.

The term “low level of serum EPA” means less than about 1.0% serum EPA (percent by weight) as shown in FIG. 1 attached hereto.

“Lysophospholipids” are derived from glycerophospholipids, by deacylation of the sn-2 position fatty acid. Lysophospholipids include, e.g., lysophosphatidic acid [“LPA”], lysophosphatidylcholine [“LPC”], lysophosphatidyletanolamine [“LPE”], lysophosphatidylserine [“LPS”], lysophosphatidylglycerol [“LPG”] and lysophosphatidylinositol [“LPI”].

The term “lipoprotein associated-phospholipase A₂” [“Lp-PLA₂”] is among the multiple cardiovascular biomarkers that have been associated with increased cardiovascular disease risk. Recently, Lp-PLA₂ has been proposed as a novel biomarker for the presence of, or impending formation of, rupture-prone plaques. Lp-PLA₂ is a member of a family of intracellular and secretory phospholipase enzymes that are capable of hydrolyzing the sn-2 ester bond of phospholipids of cell membranes and lipoproteins. Lp-PLA₂ attached to low-density lipoproteins [“LDL”] is the enzyme solely responsible for the hydrolysis of oxidized phospholipid on the LDL particle. It differs from other phospholipase enzymes in that its activity is calcium independent and it lacks activity against the naturally occurring phospholipids present in the cellular membrane.

The term “normal range” as it refers to Lp-PLA₂ is about equal or slightly less than 200 ng/mL; values higher than this place a subject at increased risk for cardiovascular events. More specifically, many commercial laboratories consider Lp-PLA₂ values between 200-235 ng/mL to be considered as borderlined high and values>235 ng/mL to be considered high. A determination that the Lp-PLA₂ levels are within “normal range” will be in accordance with the scientific understanding at the time, and not on absolute numerical values.

The term “normal subject” means an individual or person who is not taking a dyslipidemic agent(s). A dyslipidemic agent includes, but is not limited to, statins (also known as 3-hydroxy-3-methyl glutaryl coenzyme A [“HMG-CoA”] inhibitors, niacins, fibric acid derivatives and the like. More specifically, non-limiting examples of commercially available statins include: atorvastatin, cerivastatin, fluvastatin, lovastatin, mevastatin, pitavastatin, pravastatin, rosuvastatin and simvastatin. Likewise, non-limiting examples of commercially available fibric acid derivatives include: fenofibrate, bezafibrate, clofibrate and gemfibrozil, For the purposes of the present disclosure, the terms “normal” and “normal healthy” are used interchangeably herein.

“Cardiovascular disease” [“CVD”] is a broad term that encompasses a variety of diseases and conditions. It refers to any disorder in any of the various parts of the cardiovascular system. Diseases of the heart may include coronary artery disease, coronary heart disease [“CHD”], cardiomyopathy, valvular heart disease, pericardial disease, congenital heart disease (e.g., coarctation, atrial or ventricular septal defects), and heart failure. Diseases of the blood vessels may include arteriosclerosis, atherosclerosis, hypertension, stroke, vascular dementia, aneurysm, peripheral arterial disease, intermittent claudication, vasculitis, venous incompetence, venous thrombosis, varicose veins, and lymphedema. Some patients may have received treatment for their CVD, such as vascular or coronary revascularizations (angioplasty with or without stent placement, or vascular grafting). Some types of cardiovascular disease are congenital, but many are acquired later in life and are attributable to unhealthy habits, such as a sedentary lifestyle and smoking. Some types of CVD can also lead to further heart problems, such as angina, major adverse cardiovascular events [“MACEs”] and/or major coronary events [“MCEs”] such as myocardial infarction [“MI”] or require coronary intervention (i.e., coronary revascularization, angioplasty, percutaneous transluminal coronary angioplasty, percutaneous coronary intervention, and coronary artery bypass graft), or even death (i.e., cardiac or cardiovascular), which underscores the importance of efforts to treat and prevent CVD.

Primary prevention efforts are focused on reducing known risk factors for CVD, or preventing their development, with the aim of delaying or preventing the onset of CVD, MACEs or MCEs. Secondary prevention efforts are focused on reducing recurrent CVD and decreasing mortality, MACEs or MCEs in patients with established CVD.

The term “atherosclerosis” refers to a cardiovascular disease. Atherosclerosis begins with the appearance of cholesterol-laden macrophages (foam cells) in the intima of an artery. Smooth muscle cells respond to the presence of lipid by proliferating, under the influence of platelet factors. A plaque forms at the site, consisting of smooth muscle cells, leukocytes, and further deposition of lipid; in time the plaque becomes fibrotic and may calcify. Expansion of an atherosclerotic plaque leads to gradually increasing obstruction of the artery and ischemia of tissues supplied by it. Ulceration, thrombosis, or embolization of a plaque, or intimal hemorrhage and dissection, can cause more acute and severe impairment of blood flow, with the risk of infarction. In general, atherosclerosis is a cardiovascular disease in which the vessel wall is remodeled, compromising the lumen of the vessel. The atherosclerotic remodeling process involves accumulation of cells, both smooth muscle cells and monocyte/macrophage inflammatory cells, in the intima of the vessel wall. These cells take up lipid, likely from the circulation, to form a mature atherosclerotic lesion. Although the formation of these lesions is a chronic process, occurring over decades of an adult human life, the majority of the morbidity associated with atherosclerosis occurs when a lesion ruptures, releasing thrombogenic debris that precipitates events that lead to the occlusion of the artery. When such an acute event occurs in the coronary artery, myocardial infarction can ensue, and in the worst case, can result in death. Similar events can occur in the neurovascular system, leading to stroke.

The term “rupture prone-atherosclerotic plaque” and “rupture-prone lesion” are used interchangeably herein. A key characteristic of rupture-prone plaques is that the fibrous cap over the lipid core has thinned to less than about 65 μm.

The term “normal level” as it refers to triglycerides means equal to or less than about 150 mg/dL, in accordance with the current scientific understanding. Accordingly, “normal levels” of triglycerides should be determined in accordance with the scientific understanding at the time, and not on absolute numerical values.

“Lipoproteins” refer to particles whose function is to transport water-insoluble lipids and cholesterol through the body in the blood. Lipoproteins are larger and less dense, if they consist of more fat than of protein. In general, five different classes of lipoproteins are generally recognized, including: 1) chylomicrons which carry triglycerides from the intestines to the liver, skeletal muscle, and to adipose tissue; 2) very low density lipoproteins [“VLDL”] which carry (newly synthesized) triacylglycerol from the liver to adipose tissue; 3) intermediate density lipoproteins [“IDL”] which are intermediate between VLDL and LDL and not usually detectable in the blood; 4) low density lipoproteins [“LDL”] which carry cholesterol from the liver to cells of the body (also commonly referred to as the “bad cholesterol” lipoprotein); and, 5) high density lipoproteins [“HDL”] which collect cholesterol from the body's tissues and bring it back to the liver (also commonly referred to as the “good cholesterol” lipoprotein).

Thus, the term LDL refers to low density lipoproteins. Low-density lipoprotein [“LDL”] is a type of lipoprotein that transports cholesterol and triglycerides from the liver to peripheral tissues. LDL is one of the five major groups of lipoproteins (supra), although some alternative organizational schemes have been proposed. Like all lipoproteins, LDL enables fats and cholesterol to move within the water-based solution of the blood stream. LDL also regulates cholesterol synthesis at these sites. It is used medically as part of a cholesterol blood test, and since high levels of LDL cholesterol can signal medical problems like cardiovascular disease, it is sometimes called “bad cholesterol” (as opposed to HDL, which is frequently referred to as “good cholesterol” or “healthy cholesterol”).

Small dense LDL (sdLDL) Small, dense LDL is a type of LDL that is smaller and heavier than typical LDL cholesterol found in your blood. It is believed that the presence of this type of LDL can greatly increase the risk of developing atherosclerosis, which results in the formation of plaques that can accumulate to the point that they can limit—or even obstruct—blood from flowing to vital organs in the body. Because of this, having high levels of small, dense LDL may increase the risk of having a heart attack, stroke, or other form of cardiovascular disease.

A “high level of LDL” means equal to or greater than about 130 mg/dl and corresponds to those classified as having a moderate cardiovascular risk based the National Cholesterol Education Project Adult Treatment Panel III [“ATPIII”] guidelines as discussed in Davidson et al., Am. J. Cardiology, 101[suppl]:S51-S57 (2008) and shown in FIG. 1 of Davidson et al. (which reflects the current scientific understanding). The guidelines published in 2001 allowed the use of inflammatory markers as an adjunct to traditional risk factor assessments to help identify which moderate-risk individuals should be reclassified as high risk, thereby justifying reduction in the LDL cholesterol goal from less than 130 mg/dL (moderate risk) to less than 100 mg/dL (FIG. 1, Davidson et al.). As was noted above, what constitutes a “high level of LDL” should be determined in accordance with the scientific understanding at the time, and not on absolute numerical values.

The term “low in saturated fatty acids” means that the level of saturated fatty acids is equal to or less than about 15% (as a percent of total oil). More preferably, the level of saturated fatty acids is less than about 10% of the total oil composition. As was noted above, this should be determined in accordance with the scientific understanding at the time, and not on absolute numerical values.

“Arachidonic acid” [“ARA”] is the common name for cis-5,8,11,14-eicosatetraenoic acid. This fatty acid is a 20:4 omega-6 fatty acid. The term ARA as used in the present disclosure will refer to the acid or derivatives of the acid (e.g., glycerides, esters, phospholipids, amides, lactones, salts or the like) unless specifically mentioned otherwise.

The term “Inflammatory Index” refers to the ratio of the serum level of ARA to the serum level of EPA (i.e., the ARA/EPA ratio).

The term “Total Omega-3 Score™” refers to the Omega-3 Index. The Omega-Score™ is a diagnostic test that compares the levels of long-chain polyunsaturated omega-3 fatty acids (i.e., EPA and DHA) in a subject's blood to four established cut-offs for blood levels of long-chain omega-3 fatty acids in published peer-reviewed scientific journals such as Albert et al., New. Engl. J. Med., 346:1113-1118 (2002), Simon et al., Am. J. Epidemiol., 142:469-476 (1995), Lemaitre et al., Am. J. Clin. Nutr., 77:319-325 (2003), von Schacky, C. and Harris, J. Cardiovasc. Med. Suppl., 8:S46-S49 (2007).

The term “dietary supplement” refers to a product that: (i) is intended to supplement the diet and thus is not represented for use as a conventional food or as a sole item of a meal or the diet; (ii) contains one or more dietary ingredients (including, e.g., vitamins, minerals, herbs or other botanicals, amino acids, enzymes and glandulars) or their constituents; (iii) is intended to be taken by mouth as a pill, capsule, tablet, or liquid; and, (iv) is labeled as being a dietary supplement.

As used herein the term “biomass” refers specifically to spent or used yeast cellular material from the fermentation of a recombinant production host producing EPA in commercially significant amounts, wherein the preferred production host is a recombinant strain of the oleaginous yeast, Yarrowia lipolytica. The biomass may be in the form of whole cells, whole cell lysates, homogenized cells, partially hydrolyzed cellular material, and/or partially purified cellular material (e.g., microbially produced oil).

The term “lipids” refer to any fat-soluble (i.e., lipophilic), naturally-occurring molecule. A general overview of lipids is provided in U.S. Pat. Appl. Pub. No. 2009-0093543-A1 (see Table 2 therein).

The term “total lipid content” of cells is a measure of TFAs as a percent of the dry cell weight [“DCW”], although total lipid content can be approximated as a measure of FAMEs as a percent of the DCW [“FAMEs % DCW”]. Thus, total lipid content [“TFAs % DCW”] is equivalent to, e.g., milligrams of total fatty acids per 100 milligrams of DCW.

The concentration of a fatty acid in the total lipid is expressed herein as a weight percent of TFAs [“% TFAs”], e.g., milligrams of the given fatty acid per 100 milligrams of TFAs. Unless otherwise specifically stated in the disclosure herein, reference to the percent of a given fatty acid with respect to total lipids is equivalent to concentration of the fatty acid as % TFAs (e.g., % EPA of total lipids is equivalent to EPA % TFAs).

In some cases, it is useful to express the content of a given fatty acid(s) in a cell as its weight percent of the dry cell weight [“% DCW”]. Thus, for example, eicosapentaenoic acid % DCW would be determined according to the following formula: (eicosapentaenoic acid % TFAs)*(TFAs % DCW)/100. The content of a given fatty acid(s) in a cell as its weight percent of the dry cell weight [“% DCW”] can be approximated, however, as: (eicosapentaenoic acid % TFAs)*(FAMEs % DCW)/100.

The terms “lipid profile” and “lipid composition” are interchangeable and refer to the amount of individual fatty acids contained in a particular lipid fraction, such as in the total lipid or the oil, wherein the amount is expressed as a weight percent of TFAs. The sum of each individual fatty acid present in the mixture should be 100.

The term “extracted oil” refers to an oil that has been separated from other cellular materials, such as the microorganism in which the oil was synthesized. Extracted oils are obtained through a wide variety of methods, the simplest of which involves physical means alone. For example, mechanical crushing using various press configurations (e.g., screw, expeller, piston, bead beaters, etc.) can separate oil from cellular materials. Alternately, oil extraction can occur via treatment with various organic solvents (e.g., hexane), via enzymatic extraction, via osmotic shock, via ultrasonic extraction, via supercritical fluid extraction (e.g., CO₂ extraction), via saponification and via combinations of these methods. An extracted oil does not require that it is not necessarily purified or further concentrated. The extracted oils described herein will comprise at least about 30 EPA % TFAs.

The term “blended oil” refers to an oil that is obtained by admixing, or blending, the extracted oil described herein with any combination of, or individual, oil to obtain a desired composition. Thus, for example, types of oils from different microbes can be mixed together to obtain a desired PUFA composition. Alternatively, or additionally, the PUFA-containing oils disclosed herein can be blended with fish oil, vegetable oil or a mixture of both to obtain a desired composition.

The terms “reduce” and “increase” in accordance with the methods disclosed herein are intended to mean a statistically significant reduction or increase in accordance with its general and customary meaning.

The major essential fatty acids in the diet are linoleic acid (18:2) [“LA”], an omega-6 fatty acid, and alpha-linolenic acid (18:3) [“ALA”], an omega-3 fatty acid. These fatty acids have their first double bond at the 6^{th} or 3^{rd} carbon position from the omega or methyl end of the fatty acid chain, respectively. The human body cannot place a double bond at these positions. LA is converted to arachidonic acid (20:4, omega-6) [“ARA”], which can have prothrombotic and proinflammatory effects. The major omega-3 fatty acids in the diet are ALA (found in plant oils such as flax seed oil, canola oil, and soybean oil), EPA and DHA, which can be made from ALA or eaten directly as found in fish and fish oil. EPA has been reported to have antithrombotic and anti-inflammatory effects. Elevated plasma levels of phospholipid DHA have been linked to a decreased risk of dementia and Alzheimer's Disease (Schaefer, E. J. et al., Arch. Neurol., 63:1545-1550 (2006)). High doses of fish oil have been shown to be very effective for lowering plasma triglyceride levels, and reducing the secretion of very low density lipoprotein apolipoprotein B-100 (Phillipson, B. E. et al., N. Engl. J. Med., 312:1210-1216 (1985); Chan, D. C. et al., Am. J. Clin. Nutr., 77:300-307 (2003)). It has also been documented that high doses of fish oil will reduce tumor necrosis factor [“TNF”] alpha and that diets high in fish will reduce cell mediated immunity in humans (Endres, S., et al., N. Eng. J. Med., 320:265-271 (1989); Meydani, S. N. et al., J. Clin. Invest., 92:105-113 (1993)).

Studies in the United Kingdom have documented beneficial effects of fish consumption or the use of two fish oil capsules per day in reducing coronary heart disease [“CHD”] death by 29% in over 2000 patients with established CHD (Burr, M. L. et al., Lancet, 2:757-761 (1989)). However this was not confirmed in a followup study (Burr, M. L., Proc. Nutr. Soc., 66:9-15 (2007)). In the large Italian study (“Gruppo Italiano per lo Studio della Sopravvivenza nell'Infarto” or “GISSI”) in over 10,000 post-myocardial infarction patients, the use of 1 gram per day of concentrated fish oil (containing 465 mg of EPA and 375 mg of DHA) was associated with a reduction in overall recurrence of CHD, and a very striking 53% reduction in sudden death in the first 4 months after myocardial infarction in those receiving the active supplement (GISSI Prevenzione Investigators, Lancet, 354:447-455 (1999); Marchioli, R. et al., Circulation, 105:1897-1903 (2002)). In this study no benefit of vitamin E was noted (GISSI Prevenzione Investigators, Lancet, supra).

As previously mentioned, the Japan EPA Lipid Intervention Study [“JELIS”] was designed to test the hypothesis that 1800 mg/day of EPA plus statin would reduce cardiovascular risk in Japanese subjects who had elevated baseline total blood cholesterol of over 250 mg/dl (Yokoyama, M. et al., supra). In this study 15,000 subjects without CHD (4,204 men and 10,796 women) and 3,645 subjects with CHD (1,656 men and 1,989 women), between 40 and 75 years of age, were all placed on statin and then randomized to an open label, endpoint blinded manner to an EPA 1800 mg/day group or a control group (Matsuzaki, M. et al., Circ J., 73:1283-1290 (2009)). The primary endpoint was major cardiovascular event (sudden death, fatal or non-fatal myocardial infarction, unstable angina, angioplasty or coronary artery bypass surgery). After 4.6 years of follow-up, there were 9,326 who received EPA plus statin and 9,319 in the control “statin-only” group, and 262 events (2.8%) were observed in the EPA plus statin group versus 324 events (3.5%) in the control group (relative risk reduction of 19%, p=0.011). No significant differences in sudden death rates between the groups were noted.

In the patients with a history of prior CHD, events were also reduced 19% (event rates of 8.7% versus 10.7%) by EPA plus statin versus the statin-only treatment (p=0.048, number needed to treat=49) (Matsuzaki, M. et al., supra). In 1,050 subjects with a history of prior myocardial infarction, risk of subsequent CHD events was reduced by EPA plus statin by 27% from 20.0% to 15.0%, p=0.033, with a number needed to treat to prevent one event being only 19. Risk reduction in subjects without CHD was 18% with event rates of 1.4% versus 1.7%, but the p value was 0.132 (not significant). Use of EPA plus statin in JELIS was not associated with a significant reduction in stroke (1.3% versus 1.5%) for the entire cohort (Tanaka, K. et al., Stroke, 39:2052-2058 (2008)). However, for those subjects with prior stroke, use of EPA plus statin was associated with a 20% relative risk reduction in stroke (6.8% versus 10.5%, p<0.05) (Tanaka, K. et al., supra).

The most striking effect on CHD risk reduction benefit was noted in those subjects with triglyceride levels>150 mg/dl and high density lipoprotein [“HDL”] cholesterol levels<40 mg/dl (Saito, Y., et al., Atherosclerosis, 200:135-140 (2008)). In this group, the risk of developing CHD on trial was increased 1.71 as compared to statin-only controls, and the use of EPA plus statin in this group reduced CHD events by 53% (p=0.043). The most recent subgroup analysis of JELIS was carried out in subjects with impaired glucose tolerance (fasting glucose>110 mg/dl) (Oikawa, S. et al., Atherosclerosis, 206:535-539 (2009)). In this group, this risk was increased 1.63 versus the statin-only controls, and EPA plus statin reduced their risk by 22% (p=0.048), versus 18% in the normal glucose group (not significant). The use of statin resulted in a 25% mean reduction in low density lipoprotein [“LDL”] cholesterol level as compared to baseline, but the use of EPA plus statin was not associated with any significant effects on lipid levels (Yokoyama, M. et al., supra; Matsuzaki, M. et al., supra; Tanaka, K. et al., supra; Saito, Y., et al., supra; Oikawa, S. et al., supra). The overall results indicate that EPA at a dose of 1800 mg/day plus statin is effective in reducing major cardiovascular events in patients with prior CHD and stroke, in those with impaired glucose tolerance, and especially in those with dyslipidemia, and that these effects are independent of lipid lowering. Omega-3 fatty acids have also been tested to determine whether they can prevent cardiac arrhythmias in patients with implanted cardiac defibrillators. At this time, based on three studies, there is no evidence of a significant benefit of moderate doses of omega-3 fatty acids in this circumstance (Brouwer, I. A. et al., Eur. Heart J., 30:820-826 (2009)).

The underlying mechanisms whereby EPA attenuates the atherosclerotic process are unclear, particularly as they appear to be independent of changes in traditional risk factors such as LDL. In this regard, direct anti-atherosclerotic effects may be important. One such effect could be related to lipoprotein-associated phospholipase A₂ [“Lp-PLA₂”]. This enzyme is a member of a broad family of phospholipase enzymes that hydrolyze the sn-2 ester of phospholipids. Lp-PLA₂ is unique in that its activity is calcium independent and its preferred substrate is oxidized LDL, and not the naturally occurring phospholipids commonly found in the cell membrane. Lp-PLA₂ is made and secreted by macrophages in the arterial wall. The increased production of Lp-PLA₂ destabilizes the fibrous cap leading to acute myocardial infarction and stroke. Oxidized LDL is considered to be more atherogenic than natural LDL. Lp-PLA₂ is so named as it is transported in the blood associated with LDL attached to the apolipoprotein B100 structural protein, although it can also be found associated with HDL as well. In light of this biology, Lp-PLA₂ is an emerging cardiovascular risk factor and target for therapeutic intervention. Patients presenting with Lp-PLA₂ levels>200 ng/mL are considered to be at risk and should be managed accordingly. Therapeutic approaches for managing elevated Lp-PLA₂ are very limited, but may include lipid-lowering agents such as statins, niacin, fenofibrate and omega-3 fatty acids. The relative importance of EPA versus DHA is unknown.

A goal of the present disclosure was to evaluate the effects of low (600 mg/day) and high dose (1800 mg/day) EPA, and low dose DHA (600 mg/day) versus olive oil (placebo) on cardiovascular disease risk factors in a randomized, blinded, placebo controlled fashion in normal healthy subjects. Although the safety profile of omega-3 fatty acids is considered to be excellent and these fatty acids are generally recognized as safe [“GRAS”] by the United States Food and Drug Administration when given together at doses of up to 3.0 grams/day (Bays, H. E, Am. J. Cardiol., 99(suppl.):35C-43C (2007)), historical concerns linger related to untoward impact on blood clotting parameters and LDL cholesterol. EPA and DHA were used in pure forms to enable specific assessment of these two fatty acids on LDL and Lp-PLA₂.

Accordingly, in one aspect the invention concerns a method for maintaining or lowering Lp-PLA₂ levels in a normal subject which comprises administering an effective amount of EPA.

In another aspect, the invention concerns maintaining or lowering Lp-PLA₂ levels in a subject which comprises administering an effective amount of EPA substantially free of DHA.

Preferably, the initial Lp-PLA₂ levels are in the normal (i.e., equal to or slightly less than 200 ng/mL) or borderline high (i.e., between 200-235 ng/L) range. Values higher than normal place a subject at increased risk for cardiovascular events.

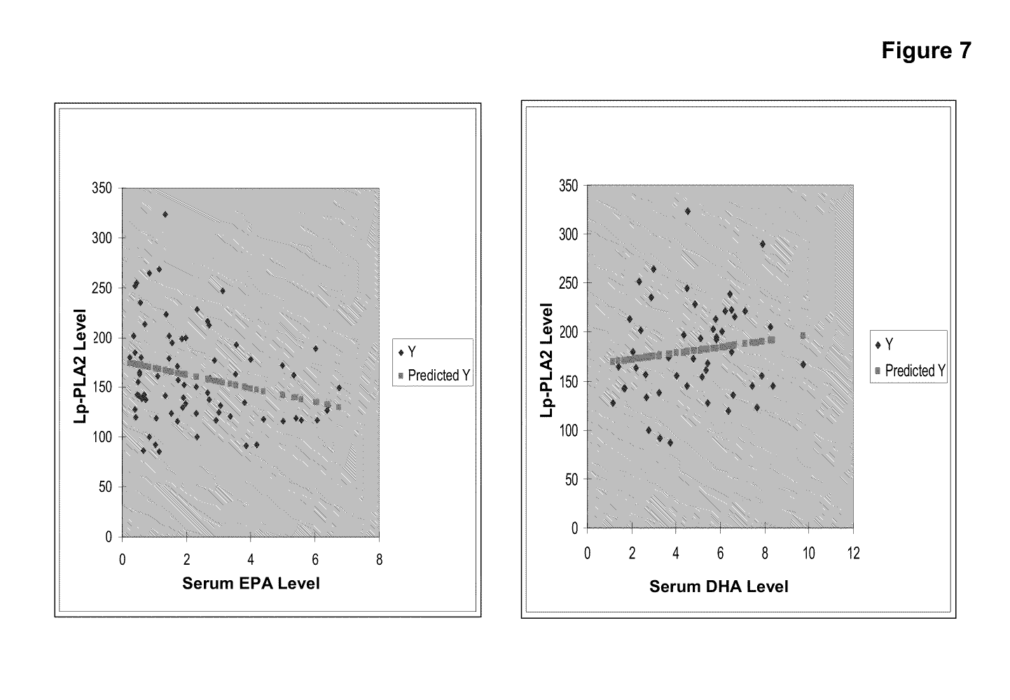

The regression analysis set forth in FIG. 7 attached hereto shows that EPA has a statistically significant effect on Lp-PLA₂, but DHA does not. In other words, while omega-3 fatty acids, as a class of fatty acids, have been shown to lower Lp-PLA₂, the regression analysis performed in this study shows that EPA, not DHA, is the active fatty acid.

It is also observed that while omega-3 fatty acids, in conjunction with dyslipidemic agents, have been shown to lower Lp-PLA₂, previous data have been collected in patients presenting with cardiovascular disease and not in normal healthy volunteers. Accordingly, the observations set forth herein made using normal, healthy volunteers sets a precedent for using EPA as a preventative or pre-emptive nutritional intervention to maintain Lp-PLA₂ in a normal range or lower Lp-PLA₂, preferably from a borderline high range into the normal range.

Thus, in another embodiment, the invention concerns a method for pre-emptive intervention in maintaining or lowering Lp-PLA₂ levels without raising LDL cholesterol levels in a normal subject having a low serum level of EPA which comprises administering an effective amount of EPA.

In another aspect, the invention concerns a method for pre-emptive intervention in maintaining or lowering Lp-PLA₂ levels without raising LDL cholesterol levels in a subject having a low serum level of EPA which comprises administering an effective amount of EPA that is substantially free of DHA.

While omega-3 fatty acids administered as LOVAZA™ [U.S. Pat. No. 5,502,077, No. 5,656,667 and No. 5,698,594], comprising both EPA and DHA, have been used to lower Lp-PLA₂, such a combination carries with it an attendant risk that LDL cholesterol will be raised, particularly in patients presenting with elevated TG. In contrast, EPA does not pose such a risk.

It should also be noted that while Lp-PLA₂ is commonly found on LDL and so it is perhaps not unexpected to see a reduction in Lp-PLA₂ with cholesterol lowering agents (e.g., statins and fibrates), in the disclosure herein, the decrease in Lp-PLA₂ occurred in the absence of any reduction in LDL.

Accordingly, in another aspect, the invention concerns a method for maintaining or lowering Lp-PLA₂ levels without raising LDL cholesterol levels in a normal subject which comprises administering an effective amount of EPA.

Still further, the invention also concerns a method for maintaining or lowering Lp-PLA₂ levels without raising LDL cholesterol levels in a subject which comprises administering an effective amount of EPA substantially free of DHA.

Any type of EPA-rich oil can be used in the method of the invention provided that if some amount of DHA is also present in the EPA-rich oil, then the amount of DHA should be such that it does not interfere with achieving any of the desired effects set forth herein. A preferred EPA-rich oil for use in the present invention is substantially free of DHA.

As will be well known to one of skill in the art, multiple sources of EPA-rich oil are commercially available. In addition to the microbial-sourced EPA oil described herein from Yarrrowia lipolytica, one could also use other EPA sources such as Epadel®, a high purity EPA ethyl ester manufactured and sold by Mochida Pharmaceutical Co., Ltd. (U.S. Pat. No. 7,498,359). This oil is indicated for hyperlipidemia and arteriosclerosis obliterans.

The EPA oil substantially free of DHA that was used in the clinical study described in Example 4 of the present disclosure was obtained from genetically modified oleaginous yeast. Specifically, the oleaginous yeast Yarrowia lipolytica was used. Oleaginous yeast are defined as those yeast that are naturally capable of oil synthesis and accumulation, wherein oil accumulation is at least 25% of the cellular dry weight. Preferably, EPA is in a triglyceride form.

U.S. Pat. Appl. Pub. No. 2009-0093543-A1 describes optimized recombinant Yarrowia lipolytica strains having the ability to produce microbial oils comprising at least about 43.3 EPA % TFAs, with less than about 23.6 LA % TFAs (an EPA:LA ratio of 1.83) and less than about 9.4 oleic acid (18:1) % TFAs. The preferred strain was Y4305, whose maximum production was 55.6 EPA % TFAs, with an EPA:LA ratio of 3.03. Generally, the EPA strains of U.S. Pat. Appl. Pub. No. 2009-0093543-A1 comprised the following genes of the omega-3/omega-6 fatty acid biosynthetic pathway: a) at least one gene encoding delta-9 elongase; and, b) at least one gene encoding delta-8 desaturase; and, c) at least one gene encoding delta-5 desaturase; and, d) at least one gene encoding delta-17 desaturase; and, e) at least one gene encoding delta-12 desaturase; and, f) at least one gene encoding C_{16/18} elongase; and, g) optionally, at least one gene encoding diacylglycerol cholinephosphotransferase [“CPT1”]. Since the pathway is genetically engineered into the host cell, there is no DHA concomitantly produced due to the lack of the appropriate enzymatic activities for elongation of EPA to DPA (catalyzed by a C_{20/22} elongase) and desaturation of DPA to DHA (catalyzed by a delta-4 desaturase). The disclosure also described microbial oils obtained from these engineered yeast strains and oil concentrates thereof.

More recently, U.S. Provisional Pat. Appl. No. 61/187,366 (filed Jun. 16, 2009, having E.I. du Pont de Nemours & Co., Inc. Attorney Docket Number CL4674) and U.S. Provisional Pat. Appl. No. 61/187,368 (filed Jun. 16, 2009, having E.I. du Pont de Nemours & Co., Inc. Attorney Docket Number CL4714) teach optimized strains of recombinant Yarrowia lipolytica having the ability to produce further improved microbial oils relative to those strains described in U.S. Pat. Appl. Pub. No. 2009-0093543-A1, based on the EPA % TFAs and the ratio of EPA:LA. In addition to expressing genes of the omega-3/omega-6 fatty acid biosynthetic pathway as detailed in U.S. Pat. Appl. Pub. No. 2009-0093543-A1, these improved strains are distinguished by: a) comprising at least one multizyme, wherein said multizyme comprises a polypeptide having at least one fatty acid delta-9 elongase linked to at least one fatty acid delta-8 desaturase [a “DGLA synthase”]; and, b) optionally comprising at least one polynucleotide encoding an enzyme selected from the group consisting of a malonyl CoA synthetase or an acyl-CoA lysophospholipid acyltransferase [“LPLAT”]; and, c) comprising at least one peroxisome biogenesis factor protein whose expression has been down-regulated; and, d) producing at least about 50 EPA % TFAs; and, e) having a ratio of EPA:LA of at least about 3.1.

Specifically, in addition to possessing at least about 50 EPA TFAs, the lipid profile within the improved optimized strains of Yarrrowia lipolytica of U.S. Provisional Pat. Appls. No. 61/187,366 and No. 61/187,368, or within extracted or unconcentrated oil therefrom, will have a ratio of EPA % TFAs to LA % TFAs of at least about 3.1. Lipids produced by the improved optimized recombinant Y. lipolytica strains are also distinguished as having less than 0.5% GLA or DHA (when measured by GC analysis using equipment having a detectable level down to about 0.1%) and having a saturated fatty acid content of less than about 8%. This low percent of saturated fatty acids (i.e., 16:0 and 18:0) results in substantial health benefits to humans and animals.

Thus, it is considered that the EPA oils described above from genetically engineered strains of Yarrowia lipolytica are substantially free of DHA, in a triglyceride form and low in saturated fatty acids.

EPA delivered as a triglyceride provides the fatty acid in a natural form that is delivered directly into the blood stream via the thoracic duct leading to a potentially more rapid onset of action. In contrast, EPA delivered as an ethyl ester must first go to the liver via the portal vein where it is subject to hepatic metabolism and then released into the blood stream. In this regard, the triglyceride form of EPA may be a preferred way to deliver EPA, resulting in less oil being needed to achieve the same clinical outcome.

More specifically, EPA in its triglyceride form is digested in the small intestine by the emulsifying action of bile salts and the hydrolytic activity of pancreatic lipase (Carlier H., et al., Reprod. Nutr. Dev., 31:475-500 (1991); Fave G. et al., Cellular and Molecular Biology, 50(7):815-831 (2004)). The hydrolysis of a triglyceride [“TG”] molecule produces two free fatty acids [“FFAs”] and a monoglyceride. These metabolic products are then absorbed by intestinal enterocytes and reassembled again as TGs. Carrier molecules called chylomicrons then transport the TGs into the lymphatic channel and finally into the blood (Lambert, M. S. et al., Br. J. Nutr., 76:435-445 (1997)).

The digestion of EPA in its ethyl ester [“EE”] form is slightly different that that of EPA in its TG form, due to the lack of a glycerol backbone (Carlier, H. et al., supra). The small intestine pancreatic lipase hydrolyzes the fatty acids from the ethanol backbone; however, the fatty acid-ethanol bond is ˜10-50 times more resistant to pancreatic lipase as compared to hydrolysis of TGs (Yang L. Y. et al., J Lipid Res., 31(1):137-147 (1990); Yang L. Y. et al., Biochem Cell Biol., 68:480-91 (1990)). The EEs that get hydrolyzed produce FFAs and ethanol. The FFAs are taken up by the enterocytes and must be reconverted to TGs to be transported in the blood. While the TG form of EPA oils contain their own monoglyceride substrate, EE oils do not. Thus, EE must therefore obtain a monoglyceride substrate from another source, thereby possibly delaying re-synthesis of TGs. This may suggest that transport to the blood is more efficient in natural TG oils in comparison to EE oils.

Numerous studies have assessed the absorption and bioavailability of TG versus EE fish oils. Most studies have measured the amount of EPA and DHA in blood plasma after ingestion of fatty acids as either TGs or EEs. Although a few studies have found that the absorption rate is similar between the two types of oils, the overall evidence suggests that TG fish oils are better absorbed in comparison to EEs. Natural TG fish oil results in 50% more plasma EPA and DHA after absorption in comparison to EE oils (Beckermann B., et al., Arzneimittelforschung, 40(6):700-704 (1990)); TG forms of EPA and DHA were shown to be 48% and 36% better absorbed than EE forms (Lawson L. D. and B. G. Hughes. Biochem. Biophys. Res. Commun., 52:328-335 (1988)); EPA incorporation into plasma lipids was found to be considerably smaller and took longer when administered as an EE (el Boustani, S. et al., Lipids, 10:711-714 (1987)); plasma lipid concentrations of EPA and DHA were significantly higher with daily portions of salmon in comparison to 3 capsules of EE fish oil (Visioli, F. et al., Lipids, 38:415-418 (2003)); and, in the rat, DHA TG supplementation led to higher plasma and erythrocyte DHA content than did DHA EE (Valenzuela, A. et al., Ann. Nutr. Metab., 49:49-53 (2005)) and a higher lymphatic recovery of EPA and DHA (Ikeda, I. et al., Biochim. Biophys. Acta, 1259:297-304 (1995)). Additional studies that provide further evidence which suggests that omega-3 fatty acids in the natural form of TGs are more efficiently digested can be found in the following citations: Hong, D. D. et al., Biochim. Biophys. Acta, 1635(1):29-36 (2003); Hansen, J. B. et al., Eur. J. Clin. Nutr., 47:497-507 (1993); Krokan, H. E. et al., Biochim. Biophys. Acta, 1168:59-67 (1993); and, Nordøy, A. et al., Am. J. Clin. Nutr., 53:1185-90 (1991).

In another embodiment, the invention concerns a method for stabilizing a rupture prone-atherosclerotic lesion in a normal subject having a low level of serum EPA which comprises administering an effective amount of EPA. Preferably, the subject has a normal level of triglycerides; alternately or additionally, the subject may have a high level of LDL.

Also of interest is a method for stabilizing a rupture prone-atherosclerotic lesion in a subject having a low level of serum EPA which comprises administering an effective amount of EPA substantially free of DHA. Preferably, the subject has a normal level of triglycerides; alternately or additionally, the subject may have a high level of LDL.

The degree to which Lp-PLA₂ is elevated in an individual may be related to the inflammatory status of their artery walls. Lp-PLA₂ is a vascular-specific inflammatory biomarker; thus, in this regard, it may be valuable to pre-emptively treat subjects presenting with high Inflammatory Index (i.e., ARA/EPA ratio).

In another aspect, the invention concerns a method for decreasing the Inflammatory Index in a normal subject which comprises administering an effective amount of EPA.

In yet another aspect, the invention concerns a method for decreasing the Inflammatory Index in a subject which comprises administering an effective amount of EPA substantially free of DHA.

The serum ratio of ARA/EPA shows that the EPA-rich oil utilized in the clinical study described in Example 4 caused a dose-related decrease in the Inflammation Index. In contrast, the DHA-rich oil had no such effect on the Inflammation Index.

The degree to which Lp-PLA₂ is elevated in an individual may also be related to their Omega-3 Score™ status. In this regard, it may be valuable to pre-emptively treat subjects having a low Omega-3 Score™. In this regard, the measurement of EPA per se may be more sensitive than the Omega-3 Score™ as it is not diluted by the presence of DHA.

In another aspect, the invention concerns a method for increasing Total Omega-3 Score™ in a normal subject having a low level of serum EPA which comprises administering an effective amount of EPA.

Thus, in still another embodiment, the invention concerns a method for increasing Total Omega-3 Score™ in a subject having a low level of serum EPA which comprises administering an effective amount of EPA substantially free of DHA.

The observation that Lp-PLA₂ changes occurred in Example 4 in the absence of any changes in other inflammatory biomarkers (i.e., IL-6, CRP) or changes in vascular adhesion molecules (i.e., VCAM) and intercellular adhesion molecule (i.e., ICAM) support the premise that EPA has a direct effect on Lp-PLA₂ (likely at the transcriptional level) and is not some indirect, non-specific change associated with the general inflammatory process. This concept is consistent with Lp-PLA₂ being a vascular marker of atherosclerosis and plaque stability rather than some unspecific systemic biomarker of inflammation.

To the extent EPA is a specific transcriptional regulator of Lp-PLA₂, it may be adjunctive with other pharmacological approaches such as statins and fibrates, but without the attendant untoward additivity of side-effects commonly associated with polypharmacy.

This may also extend to the emerging small molecule inhibitors of Lp-PLA₂ such as darapladib that are now in late stage clinical development. For example, since it is believed that EPA (preferably substantially free of DHA) may affect gene expression, use of EPA (preferably substantially free of DHA) in combination with a compound such as daraplabid that functions by inhibiting Lp-PLA₂ may produce an additive or synergistic effect in regulating levels of Lp-PLA₂. Another small molecule inhibitors of Lp-PLA₂ rilapladib which is a backup candidate to daraplabid.

At this time, it is not clear whether regulation of Lp-PLA₂ is due to EPA itself (preferably substantially free of DHA), or due to a hydroxylated metabolite of EPA. Recent studies have now identified a new family of lipid anti-inflammatory mediators, termed resolvins (“resolution phase interaction products”), which are very potent as indicated by their biological activity in the low nanomolar range. Within this family are EPA-derived resolvins (i.e., E-series resolvins or “RvEs”) (reviewed in Serhan, C. N., Pharma. & Therapeutics, 105:7-21 (2005)). The distinct role of RvE1 (5S,12R,18R-trihydroxy-6Z,8E,10E,14Z,16E-EPA), as demonstrated in Arita, M. et al. (Proc. Natl. Acad. Sci. U.S.A., 102(21):7671-7676 (2005)) offers mechanistic evidence that may form the basis for some of the beneficial actions of EPA in human health and disease.

This new biology underscores the potential utility of EPA-rich products in both the nutritional and medical management of inflammatory processes. Furthermore, since inflammation underlies many diseases ranging from cardiovascular to metabolic (e.g., metabolic syndrome X, obesity, diabetes) to neurological diseases (e.g., Alzheimers), it is expected that EPA-enriched oils (such as those described herein) will have very broad utility. It is expected that medical utility may be derived from: 1) use of EPA or RvEs as bioactives in medical foods; and/or, 2) addition of EPA to over-the-counter or prescriptive medications as adjunctive therapy. Finally, EPA may find utility as a precursor for the synthesis of RvEs and medicinally-optimized new chemical entities.

In some embodiments, the claimed methods of administration for maintaining or lowering Lp-PLA₂ levels (optionally without raising LDL cholesterol levels), stabilizing a rupture prone-atherosclerotic lesion, decreasing the Inflammatory Index, and increasing Total Omega-3 Score™ is a first-line therapy, meaning that it is the first type of therapy given for the condition or disease. In other embodiments, the claimed method of administration is a second-line therapy, meaning that the treatment is given when initial treatment (first-line therapy) does not work adequately with respect to treatment goals, or ceases to be adequate, e.g. due to physiological changes in the patient or changes in CHD risk factors.

Similarly, in some embodiments, the invention is suitable for primary prevention. In other embodiments, the invention is suitable for secondary prevention.

Although the Examples demonstrate the methods disclosed herein using concentrated EPA administered orally in the dosage form of a soft-gel capsule, this should by no means be construed as a limitation to the present disclosure. For example, as is well known to one of skill in the art, EPA may be administered in a capsule, a tablet, granules, a powder that can be dispersed in a beverage, or another solid oral dosage form, a liquid (e.g., syrup), a soft gel capsule, a coated soft gel capsule or other convenient dosage form such as oral liquid in a capsule. Capsules may be hard-shelled or soft-shelled and may be of a gelatin or vegetarian source. EPA may also be contained in a liquid suitable for injection or infusion.

Additionally, EPA, preferably substantially free of DHA, may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

Excipients may include, but are not limited to, surfactants, such as propylene glycol monocaprylate, mixtures of glycerol and polyethylene glycol esters of long fatty acids, polyethoxylated castor oils, glycerol esters, oleoyl macrogol glycerides, propylene glycol monolaurate, propylene glycol dicaprylate/dicaprate, polyethylene-polypropylene glycol copolymer, and polyoxyethylene sorbitan monooleate, cosolvents such ethanol, glycerol, polyethylene glycol, and propylene glycol, and oils such as coconut, olive or safflower oils. The use of surfactants, cosolvents, oils or combinations thereof is generally known in the pharmaceutical arts, and as would be understood to one skilled in the art, any suitable surfactant may be used in conjunction with the present invention and embodiments thereof.

The dose concentration, dose schedule and period of administration of the composition should be sufficient for the expression of the intended action, and may be adequately adjusted depending on, for example, the dosage form, administration route, severity of the symptom(s), body weight, age and the like. When orally administered, the composition may be administered in three divided doses per day, although the composition may alternatively be administered in a single dose or in several divided doses.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### General Methods

The meaning of abbreviations is as follows: “sec” means second(s), “min” means minute(s), “h” means hour(s), “d” means day(s), “μL” means microliter(s), “mL” means milliliter(s), “L” means liter(s), “dl” means deciliter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s), “kB” means kilobase(s), “DCW” means dry cell weight, and “TFAs” means total fatty acids.

### Example 1

Generation of Yarrowia lipolytica Strain Y4305 F1B1 to Produce about 50-52% EPA of Total Fatty Acids [“TFAs”] with 28-32% Total Lipid Content

The present Example describes the construction of strain Y4305 F1B1, derived from Yarrowia lipolytica ATCC #20362, capable of producing about 50-52% EPA relative to the total lipids with 28-32% total lipid content [“TFAs % DCW”] via expression of a Δ9 elongase/Δ8 desaturase pathway.

Strain Y4305F1B1 is derived from Yarrowia lipolytica strain Y4305, which has been previously described in the General Methods of U.S. Pat. App. Pub. No. 2008-0254191, published on Apr. 9, 2009, the disclosure of which is hereby incorporated in its entirety.

Description of Parent Strain Y4305 (Producing about 53% EPA of TFAs)

The final genotype of strain Y4305 with respect to wild type Yarrowia lipolytica ATCC #20362 was SCP2-(YALI0E01298g), YALI0C18711g-, Pex10-, YALI0F24167g-, unknown 1-, unknown 3-, unknown 8-, GPD::FmD12::Pex20, YAT1::FmD12::OCT, GPM/FBAIN::FmD12S::OCT, EXP1::FmD12S::Aco, YAT1::FmD12S::Lip2, YAT1::ME3S::Pex16, EXP1::ME3S::Pex20 (3 copies), GPAT::EgD9e::Lip2, EXP1::EgD9eS::Lip1, FBAINm::EgD9eS::Lip2, FBA::EgD9eS::Pex20, GPD::EgD9eS::Lip2, YAT1::EgD9eS::Lip2, YAT1::E389D9eS::OCT, FBAINm::EgD8M::Pex20, FBAIN::EgD8M::Lip1 (2 copies), EXP1::EgD8M::Pex16, GPDIN::EgD8M::Lip1, YAT1::EgD8M::Aco, FBAIN::EgD5::Aco, EXP1::EgD5S::Pex20, YAT1::EgD5S::Aco, EXP1::EgD5S::ACO, YAT1::RD5S::OCT, YAT1::PaD17S::Lip1, EXP1::PaD17::Pex16, FBAINm::PaD17::Aco, YAT1::YICPT1::ACO, GPD::YICPT1::ACO. The structure of the above expression cassettes are represented by a simple notation system of “X::Y::Z”, wherein X describes the promoter fragment, Y describes the gene fragment, and Z describes the terminator fragment, which are all operably linked to one another. Abbreviations are as follows: FmD12 is a Fusarium moniliforme delta-12 desaturase gene [U.S. Pat. No. 7,504,259]; FmD12S is a codon-optimized delta-12 desaturase gene, derived from Fusarium moniliforme [U.S. Pat. No. 7,504,259]; MESS is a codon-optimized C_{16/18} elongase gene, derived from Mortierella alpina [U.S. Pat. No. 7,470,532]; EgD9e is a Euglena gracilis delta-9 elongase gene [Inn App. Pub. No. WO 2007/061742]; EgD9eS is a codon-optimized delta-9 elongase gene, derived from Euglena gracilis [Intl App. Pub. No. WO 2007/061742]; E389D9eS is a codon-optimized delta-9 elongase gene, derived from Eutreptiella sp. CCMP389 [U.S. Pat. Appl. Pub. No. 2007-0117190-A1]; EgD8M is a synthetic mutant delta-8 desaturase gene [Inn App. Pub. No. WO 2008/073271], derived from Euglena gracilis [U.S. Pat. No. 7,256,033]; EgD5 is a Euglena gracilis delta-5 desaturase [U.S. Pat. App. Pub. US 2007-0292924-A1]; EgDSS is a codon-optimized delta-5 desaturase gene, derived from Euglena gracilis [U.S. Pat. App. Pub. No. 2007-0292924]; and, RDSS is a codon-optimized delta-5 desaturase, derived from Peridinium sp. CCMP626 [U.S. Pat. App. Pub. No. 2007-0271632]. PaD17 is a Pythium aphanidermatum delta-17 desaturase gene [U.S. Pat. No. 7,556,949]; PaD17S is a codon-optimized delta-17 desaturase gene, derived from Pythium aphanidermatum [U.S. Pat. No. 7,556,949]; YICPT1 is a Yarrowia lipolytica diacylglycerol cholinephosphotransferase gene [Intl App. Pub. No. WO 2006/052870].

Total lipid content of the Y4305 cells was 27.5 [“TFAs % DCW”], and the lipid profile was as follows, wherein the concentration of each fatty acid is as a weight percent of TFAs [“% TFAs”]: 16:0 (palmitate)—2.8, 16:1 (palmitoleic acid)—0.7, 18:0 (stearic acid)—1.3, 18:1 (oleic acid)—4.9, 18:2 (LA)—17.6, ALA—2.3, EDA—3.4, DGLA—2.0, ARA—0.6, ETA—1.7 and EPA—53.2.

### Generation Of Strain Y4305 F1B1

Strain Y4305 was subjected to transformation with a dominant, non-antibiotic marker for Yarrowia lipolytica based on sulfonylurea [“SU^{R}”] resistance. More specifically, the marker gene is a native acetohydroxyacid synthase (“AHAS” or acetolactate synthase; E.C. 4.1.3.18) that has a single amino acid change, i.e., W497L, that confers sulfonyl urea herbicide resistance (SEQ ID NO:292 of Intl. App. Pub. No. WO 2006/052870). AHAS is the first common enzyme in the pathway for the biosynthesis of branched-chain amino acids and it is the target of the sulfonylurea and imidazolinone herbicides.

The random integration of the SU^{R} genetic marker into Yarrowia strain Y4305 was used to identify those cells having increased lipid content when grown under oleaginous conditions relative to the parent Y4305 strain.

Specifically, a mutated AHAS gene, described above, was introduced into Yarrowia cells as a linear DNA fragment. The AHAS gene integrates randomly throughout the chromosome at any location that contains a double stranded-break that is also bound by the Ku enzymes. Non-functional genes or knockout mutations were generated when the SU^{R} fragment integrated within the coding region of a gene. Every gene is a potential target for disruption. Thus, a random integration library in Yarrowia cells was made and SU^{R} mutant cells that were identified. Candidates were evaluated based on DCW (g/L), FAMEs % DCW, EPA TFAs and EPA % DCW.

Out of the 48 mutant cultures evaluated, only three of the cultures (i.e., F1B1 [15.1 EPA % DCW], F1B5 [15.6 EPA % DCW], and F1G6 [16.1 EPA % DCW] were selected for further evaluation in triple flask analysis. The results of the triple flask analysis are summarized in Table 1.

TABLE 1
Shake Flask Evaluation Of Individual Y4305 SU^{R} Mutants

| | DCW | TFAs % | EPA % | EPA % |
|---|---|---|---|---|
| Strain | (g/L) | DCW | TFAs | DCW |
| Y4305 | 6.8 | 25.1 | 50.3 | 12.7 |
| Y4305 F1B1 | 6.9 | 27.9 | 53.1 | 14.8 |
| Y4305 F1B5 | 6.9 | 27.7 | 53.0 | 14.7 |
| Y4305 F1G6 | 7.2 | 27.8 | 52.4 | 14.6 |

Since strain Y4305-F1B1 possessed the highest EPA productivity [“EPA % DCW”] and lipid content [“TFAs % DCW”] of those evaluated, this mutant was selected for further evaluation under two liter fermentation conditions (parameters similar to those of U.S. Pat. Appl. Pub. No. 2009-009354-A1, Example 10).

Average EPA productivity [“EPA % DCW”] for strain Y4305 was 50-56, as compared to 50-52 for strain Y4305-F1B1. Average lipid content [“TFAs % DCW”] for strain Y4305 was 20-25, as compared to 28-32 for strain Y4305-F1B1. Thus, lipid content was increased 29-38% in strain Y4503-F1B1, with minimal impact upon EPA productivity.

### Example 2

Fermentation and Downstream Processing to Obtain EPA Containing Microbial Oil from Yarrowia lipolytica Strain Y4305 F1B1

Inocula were prepared from frozen cultures of Yarrowia lipolytica strain Y4305 F1B1 in a shake flask. After an incubation period, the culture was used to inoculate a seed fermentor. When the seed culture reached an appropriate target cell density, it was then used to inoculate a larger fermentor. The fermentation is a 2-stage fed-batch process. In the first stage, the yeast were cultured under conditions that promote rapid growth to a high cell density; the culture medium comprised glucose, various nitrogen sources, trace metals and vitamins. In the second stage, the yeast were starved for nitrogen and continuously fed glucose to promote lipid and PUFA accumulation. Process variables including temperature (controlled between 30-32° C.), pH (controlled between 5-7), dissolved oxygen concentration and glucose concentration were monitored and controlled per standard operating conditions to ensure consistent process performance and final PUFA oil quality.

One of skill in the art of fermentation will know that variability will occur in the oil profile of a specific Yarrowia strain, depending on the fermentation run itself, media conditions, process parameters, scale-up, etc., as well as the particular time-point in which the culture is sampled (see, e.g., U.S. Pat. Appl. Pub. No. 2009-0093543-A1).

After fermentation, the yeast biomass is dewatered and washed to remove salts and residual medium, and to minimize lipase activity. Drum drying follows to reduce the moisture to less than 5% to ensure oil stability during short term storage and transportation.

Mechanical disruption with a food grade iso-hexane solvent is then used to extract the EPA rich oil from the biomass. The cell debris is removed and the solvent is evaporated to yield a crude oil. The crude oil is degummed using phosphoric acid and refined with 20° Be caustic to remove phospholipids, trace metals and free fatty acids. Bleaching with silica and clay is used to adsorb color compounds and minor oxidation products, which is followed by winterization to remove high melting compounds that would otherwise precipitate out over the storage period. The last deodorization step strips out volatile, odorous and additional color compounds to yield the high quality EPA-rich Omega-3 oil in its natural triglyceride form. The final deodorized oil contains 35% EPA in fatty acids on the total oil basis and has a peroxide value of 0.1, an Anisidine value of 2 and an unsaponifiable level of 1.1%. Antioxidants are added at various stages of the process to ensure the oxidative stability of the EPA oil.

### Example 3

### EPA Oil Encapsulation and Packaging for Clinical Studies

In preparation of a clinical study, designed to test the safety and efficacy of the EPA-enriched oil of Example 2 as compared to an olive oil placebo and a comparator oil providing DHA (supra), four types of PUFA-containing capsules were prepared and/or packaged for human consumption.

### Oil Encapsulation

A single lot of oil from Example 2 was utilized to prepare doses of 100 mg and 300 mg EPA suitable for human consumption. Where needed, the EPA-enriched oil of Example 2 was diluted with olive oil. The same lot of olive oil was also used to prepare the control. Food-grade antioxidants designed to minimize oil degradation were added to the olive oil control (and therefore the olive oil used to dilute the EPA-enriched oil). Thus, both the 100 mg and 300 mg EPA oils contained the appropriate amount of anti-oxidant. The composition of the olive oil, 100 mg EPA oil and 300 mg EPA oil were analyzed to determine the complete fatty acid composition of each. Concentration of oleic acid (C18:1, omega-9), EPA, total saturates, total monounsaturates, total polyunsaturates and total omega-3 in each oil is shown in Table 5.

The 100 mg EPA oil, 300 mg EPA oil and olive oil control were each encapsulated in 1000 mg fill caps at Best Formulations (City of Industry, CA), using standard production equipment, protocols and testing regimes. The encapsulation material was an enteric coated, amber tinted bovine based cap material.

After encapsulation, the EPA levels and a microbial analysis was performed within a random sample of 100 mg and 300 mg EPA capsules.

### Packaging

The 100 mg EPA oil, 300 mg EPA oil and olive oil control capsules were transferred to We-Pack-It-All [“WPIA”] (Irwindale, Calif.). Separately, 100 mg DHA soft gel capsules (life's DHA™ for Kids; Martek, Columbia, Md.) were transferred to WPIA.

WPIA packaged all 4 capsule types into labeled boxes containing a week supply (i.e., 42 capsules per box). Each box contained 7 sleeves, each labeled and containing the appropriate capsules for each day of the week, with separate compartments for the 3 doses required each day, each dose consisting of 2 capsules. Specifically, boxes for the Control Group were packaged to contain 6 capsules of olive oil for each day, to be ingested at breakfast (2 capsules), lunch (2 capsules) and dinner (2 capsules), respectively. Boxes for the EPA-600 Group were packaged to contain 6 capsules of 100 mg EPA oil for each day, to be ingested at breakfast, lunch and dinner, respectively. Boxes for the EPA-1800 Group were packaged to contain 6 capsules of 300 mg EPA oil for each day, to be ingested at breakfast, lunch and dinner, respectively. Finally, boxes for the DHA Group were packaged to contain 6 capsules of 100 mg DHA oil for each day, to be ingested at breakfast, lunch and dinner, respectively. Samples of the final packaged materials were tested to confirm that the correct oil was in the correct labeled packaging.

### Example 4

### Clinical Study: A Randomized, Double-Blinded, Placebo-Controlled Study to Assess the Efficacy and Safety of EPA-Enriched Oil Derived from Yarrowia lipolytica in Healthy Subjects

The goal of this clinical study was to evaluate the effects of low (600 mg/day) and high dose (1800 mg/day) EPA, and low dose DHA (600 mg/day) versus olive oil (placebo) on cardiovascular disease risk factors in a randomized, double-blinded, placebo controlled fashion in normal healthy subjects. Although the safety profile of omega-3 fatty acids is considered to be excellent and these fatty acids are generally recognized as safe [“GRAS”] by the United States Food and Drug Administration when given together at doses of up to 3.0 grams/day (Bays, H. E, Am. J. Cardiol., 99 (suppl.) 35C-43C (2007)), historical concerns linger related to untoward impact on blood clotting parameters and LDL cholesterol. Additionally, the design of this study and use of both EPA and DHA in pure forms enables the specific assessment of these two fatty acids on LDL and Lp-PLA₂.

### A. Methods

The goal of this study was to test the safety and efficacy of an EPA-enriched oil (as described in Examples 1-3; E.I. duPont de Nemours & Co., Inc. Applied Biosciences, Wilmington, Del.), to corroborate the safety of a novel oil rich in EPA in humans prior to this product being placed on the market as a dietary supplement. This oil was tested at doses of 600 mg and 1800 mg of EPA/day as compared to olive oil placebo and a comparator omega-3 oil providing 600 mg of DHA/day over a 6 week period in a parallel arm design in approximately 120 healthy adults studied in both the fasting and post-prandial state. Safety was monitored by assessing for adverse reactions, measuring vital signs and a variety of laboratory tests including a complete metabolic profile, thyroid function tests, complete blood count, and prothrombin time.

The objective was to carry out a double blinded, randomized, placebo-controlled trial in 120 healthy subjects between 20-70 years of age over a 6 week period comparing the effects of an EPA-enriched oil provided at daily doses of EPA at 600 or 1800 mg/day compared to an oil providing 600 mg of DHA/day and an olive oil placebo. Specific parameters investigated included changes in body weight, heart rate, blood pressure, complete blood count, comprehensive metabolic profile, lipid and lipoprotein measures in the fasted and fed state, fatty acid profiles, and inflammation markers.

### A1. Study Recruitment, Eligibility, and Screening

Subjects were recruited using a computerized list of prior study participants, direct mailing and newspaper advertising. Subjects calling in to respond to letters and advertisements were screened for eligibility over the telephone. The following inclusion criteria were used: 1) healthy male or female adult volunteers with no significant chronic disease; 2) 21-70 years of age; 3) body mass index of 20-35 kg/m²; and, 4) women were required to be post-menopausal (age greater than 52 years and no menses for at least 1 year) or surgically sterile. The following exclusion criteria were used. Subjects could not be: 1) involved with competitive exercise/training; 2) be current smokers; 3) on dietary supplements that could affect serum fatty acids including fish oil, EPA or DHA, flax seed oils, weight control products, or high doses of vitamin C (>500 mg/day) or vitamin E (>400 units/day); 4) having frequent fish consumption>3 meal/week of “oily fish” such as tuna or salmon; 5) consuming >2 alcoholic drinks/day; 6) on medications which could serum lipids (such as statins, fibrates, niacin, resins, ezetimibe, hormonal replacement therapy) or body weight (medications blocking fat absorption such as Orlistat) for at least 6 weeks; and, 7) taking coumadin or more than 325 mg/day of aspirin which could effect bleeding time or the coagulation profile. Additional exclusions included: 1) a history of a bleeding disorder; 2) a history of significant cardiac, renal, hepatic, gastro-intestinal, pulmonary, neoplastic, biliary or endocrine disorders including uncontrolled thyroid disease; or, 3) uncontrolled hypertension (systolic blood pressure>160 mmHg) or diabetes (fasting glucose>200 mg/dl).

Subjects found to be eligible by telephone screening were asked to come to the clinic for a screening visit, which including signing an informed consent. The protocol used herein has been approved by a E.I. duPont de Nemours and Co., Human Studies Committee, an external IRB and registered with the National Institutes of Health at www.clinicaltrials.gov. At the screening visit all subjects were asked to fast overnight and had standard blood chemistries, and complete blood counts done. The original screening criteria were also re-checked to make sure all subjects were still eligible for this study. Subjects found to be eligible were then scheduled for an enrollment visit if they met all previously outlined entry criteria.

At all visits the following information was recorded: weight in pounds and kilograms, height in centimeters [“cm”] and inches, waist circumference in cm and inches, resting heart rate, systolic blood pressure, diastolic blood pressure, and a brief dietary assessment to assure continued lack of high fish intake and/or flax seed or fish oil dietary supplements. Blood pressure and pulse measurements were done 3 times on each visit after the subjects had been sitting quietly for 5 minutes.

### A2. Laboratory Screening and Safety Monitoring Tests

Standard chemistry tests were carried at all visits (screening, enrollment, and final study visit after an overnight fast by Quest Laboratories, Cambridge, Mass.): blood urea nitrogen, creatinine, calculated glomerular filtration rate, sodium, potassium, chloride, carbon dioxide, calcium, total protein, albumin, globulin, total bilirubin, alkaline phosphatase, liver transaminases AST and ALT, and glucose. A complete blood count was also performed at all visits and included: hemoglobin, hematocrit, red blood cell count, platelet count, white blood cell count and a white blood cell count differential. Additional tests included: prothrombin time, and measurement of thyroid function including T3, T4 and T3 uptake. All subjects entering the study were required to have: liver function tests (i.e., transaminases) of less than 3 times the upper limits of normal; bilirubin and alkaline phosphatase values in the normal range; serum creatinine levels of less than 2.5 mg/dl; hemoglobin levels over 11 g/dl; a normal prothrombin time; a fasting blood glucose below 200 mg/dl; and, a blood pressure below 170/110 mmHg. All subjects who qualified for the study and met all the screening and laboratory entry criteria were scheduled for an enrollment visit within one month of screening.

### A3. Study Capsules

At the time of the enrollment visit all subjects were randomly allocated into a protocol where they were required to take two capsules three times daily which contained a either: 1) olive oil placebo; 2) 600 mg/day of EPA/day; 3) 1800 mg of EPA/day; and, 4) 600 mg of DHA/day (Example 3). The oil composition of the capsules is provided in Table 5. The EPA oils are notable for their low levels of saturated fatty acids, particularly with regard to the DHA oil. Specifically, the composition of the DHA oil was as follows (g fatty acid per 100 g of oil): 14.1 g myristic acid (14:0), 10.8 g palmitic acid (16:0), 2 g palmitoleic acid (16.1), 7.4 g margaric acid (17:0), 8.4 g oleic acid (18:1), 0.1 g EPA (20:5n3), 0.9 g DPA (22:5n6) and 37.9 g DHA (22:6n3).

### A4. Study Protocol

At visit 2, subjects were again asked to fast for 12 hours, and information about subject characteristics including all vital signs, recent illness or hospitalization, medication and supplement use, and diet information was again obtained. Subjects then had blood drawn for a metabolic profile and complete blood counts. Thereafter, study subjects were provided with a test meal (containing 980 calories, 470 mg of cholesterol, 56 grams of fat, 20 grams of saturated fat, 0 trans fat, 70 grams of carbohydrate, and 44 grams of protein) and had a second blood drawing 4 hours after meal completion. Subjects (30 in each group) were then randomized equally for 6 weeks to one of four treatment arms: 1) olive oil placebo; 2) 600 mg of EPA/day; 3) 1800 mg of EPA/day; and, 4) 600 mg of DHA/day. At one week and three weeks after beginning the supplements, all study subjects were contacted by telephone and asked about any adverse effects and about their compliance, and the information on their calendars with regard to capsule use and fish intake. They were also asked about whether they had experienced any fishy aftertaste or odor, and if so, how frequent were these episodes, and how unpleasant were they? The entire process as listed above was repeated after study subjects were on the study capsules for 6 weeks at the time of the final visit. Compliance and adverse events were assessed by telephone at weeks 1 and 3 by telephone and at week 6 by questionnaire and capsule count. Over 42 days, subjects were expected to have consumed a total of 252 capsules. Compliance was calculated as a percentage of consumed capsule count/expected capsule count based on the number of days the subject was in the study. All subjects were asked to stay on their capsules until they come in for their final visit. Compliance in all participants who completed the study was based on capsule count was in excess of 85%, mean 96%.

### A5. Automated Laboratory Analyses

The following laboratory measurements were carried out on an automated analyzer (Roche Diagnostics, Indianapolis, Ind.) on samples obtained in the fasting state at the randomization visit, and the final visits using frozen aliquots of serum stored at −80 degrees Celcius: 1) total cholesterol; 2) triglycerides; 3) direct high density lipoprotein [“HDL”] cholesterol; 4) direct low density lipoprotein [“LDL”] cholesterol; 5) direct small dense LDL cholesterol; 6) apolipoprotein [“apo”] B; 7) apoA-I; 8) lipoprotein (a); 9) fibrinogen; 10) high sensitivity C reactive protein [“hs-CRP”]; 11) lipoprotein associated phospholipase A₂ [“Lp-PLA₂”]; and, 12) insulin as previously described. See, McNamara, J. R. and Schaefer, E. J., Clin. Chim. Acta, 166:1-8 (1987); Okada, M. et al., J. Lab. Clin. Med., 132:195-201 (1998); Hirano, T. et al., J. Lipid Res., 44:2193-2201 (2003); Ai, M. et al., Am. J. Cardiol., 101:315-318 (2008); Ingelsson, E. et al., JAMA, 298:776-785 (2007); Jenner, J. L. et al., Circulation, 87:1135-1141 (1993); Schaefer, E. J. et al., Am. J. Cardiol., 95:1025-1032 (2005); McNamara, J. R. et al., Atherosclerosis, 154:229-236 (2001).

On the samples obtained 4 hours after the fat-rich meal, total cholesterol, triglycerides, direct HDL cholesterol, and direct LDL cholesterol were measured. Direct LDL cholesterol and small dense LDL cholesterol levels were measured using kits obtained from Denka-Seiken Corporation, (Tokyo, Japan), as previously described (Okada, M. et al., supra; Hirano, T. et al., supra; Ai, M. et al., supra). Remnant lipoprotein cholesterol was measured using kits obtained from Polymedco (Cortland Manor, N.Y.) and manufactured by Otsuka Corporation (Tokyo, Japan) as previously described (McNamara, J. R. et al., Atherosclerosis, supra). All lipid assays are standardized through the Lipid Research Clinics standardization program of the Centers for Disease Control (Atlanta, Ga.). All assays had between and within run coefficients of variation of <5%. Serum fatty acid profiles were analyzed by Nutrasource Diagnostics (Guelph, Ontario, Canada).

### A6. Other Laboratory Measurements

Plasma apoB-48 was measured with an enzyme linked immunosorbent assay obtained from the Shibayagi Company (Gunma, Japan) (Kinoshita, M. et al., Clin. Chim. Acta, 351:115-120 (2005); Otokozawa, S. et al., Atherosclerosis, 205:197-201 (2009); Otokozawa, S. et al., Metabolism, 58(11): 1536-1542 (2009)). ICAM1 and VCAM1, interleukin-6 or IL-6, and adiponectin were all measured using commercially available enzyme linked immunoassays [“ELISA”] obtained from the R & D Corporation (Minneapolis, Minn.). All these assays have within and between run coefficients of variation of less than 10%.

### A7. Statistical Analyses and Hypothesis Testing

Statistical analyses compared mean absolute and percentage changes from baseline and 6 weeks in the active groups versus the placebo group. Analysis of variance, as well as paired t-test analysis were performed with SYSTAT software. P values of <0.05 are considered statistically significant. The study was run in a placebo controlled double-blinded fashion (i.e., the Principle Investigators, clinic staff, and laboratory personnel, were all blinded to the identify of the capsules and groups throughout the active portion of the study).

### A8. Study Registration and Gender/Minority Recruitment/Human Subjects

The study was registered with the National Institutes of Health at www.clinicaltrials.gov and conforms to CONSORT recommendations. The goal was to enroll 120 subjects into the study, and to have at least 100 complete the study. Information on study subjects is shown in Table 2. With regard to minority targets for this study, at least 6% African American participation, 4% Asian participation, and 8% Hispanic participation were sought, with approximately equal numbers of men and women. In actuality, there were 110 completers, with 25.5% African American participation, 2.7% Asian participation, and 1.8% Hispanic participation. Participants were 70% Caucasian and 67.3% male. Therefore, goals were met for subjects completing the study and African American participation, but fewer Asians, Hispanics, and women participated than desired. The relative lack of female participants was related to the requirement that all women be post-menopausal or surgically sterile.

### B. Results

### B1.

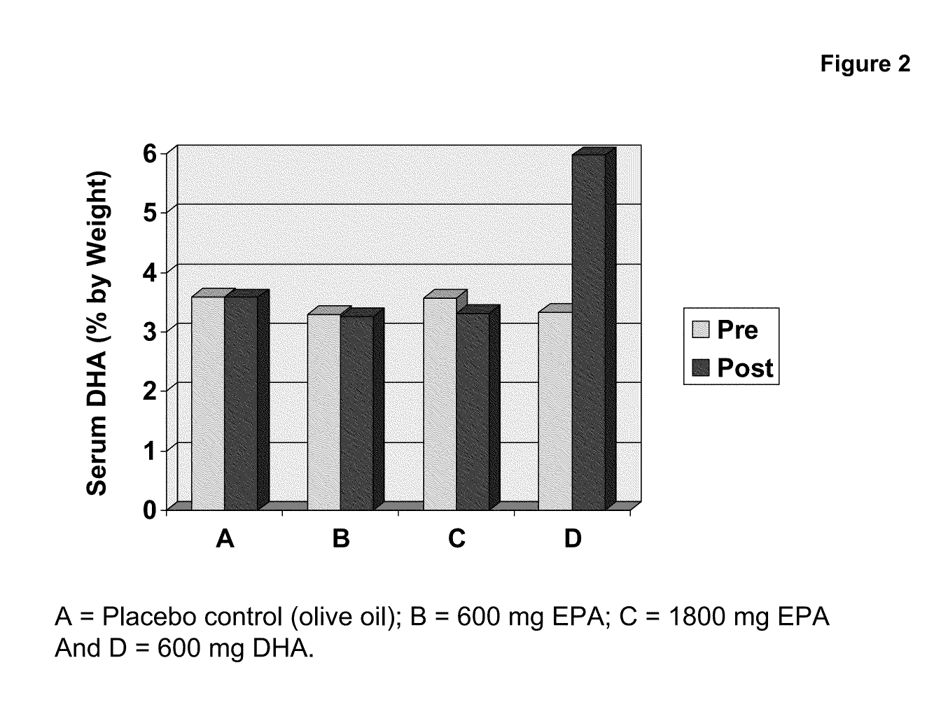

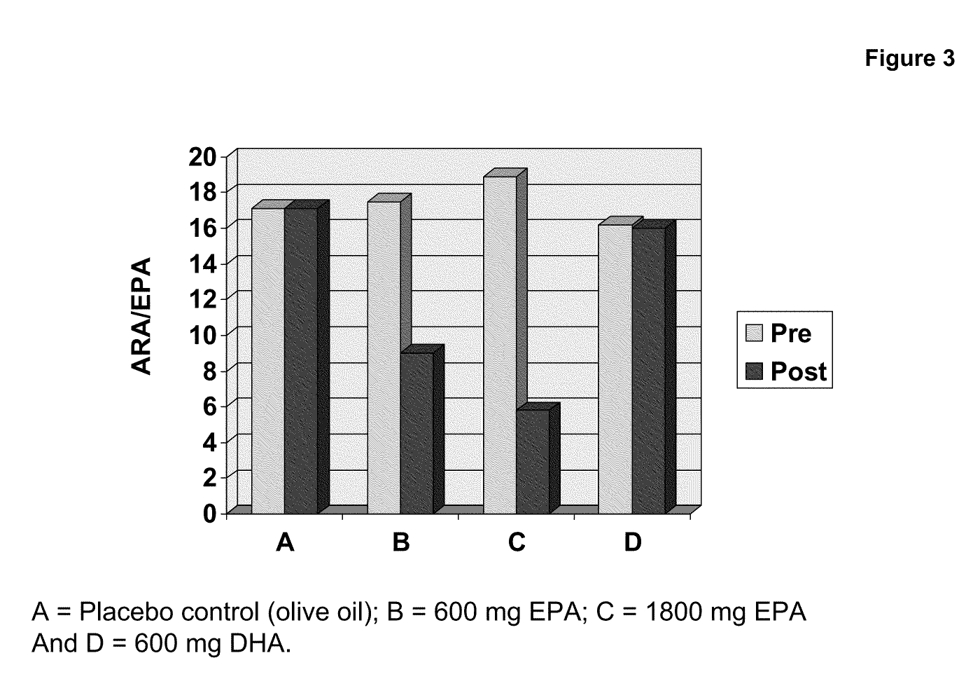

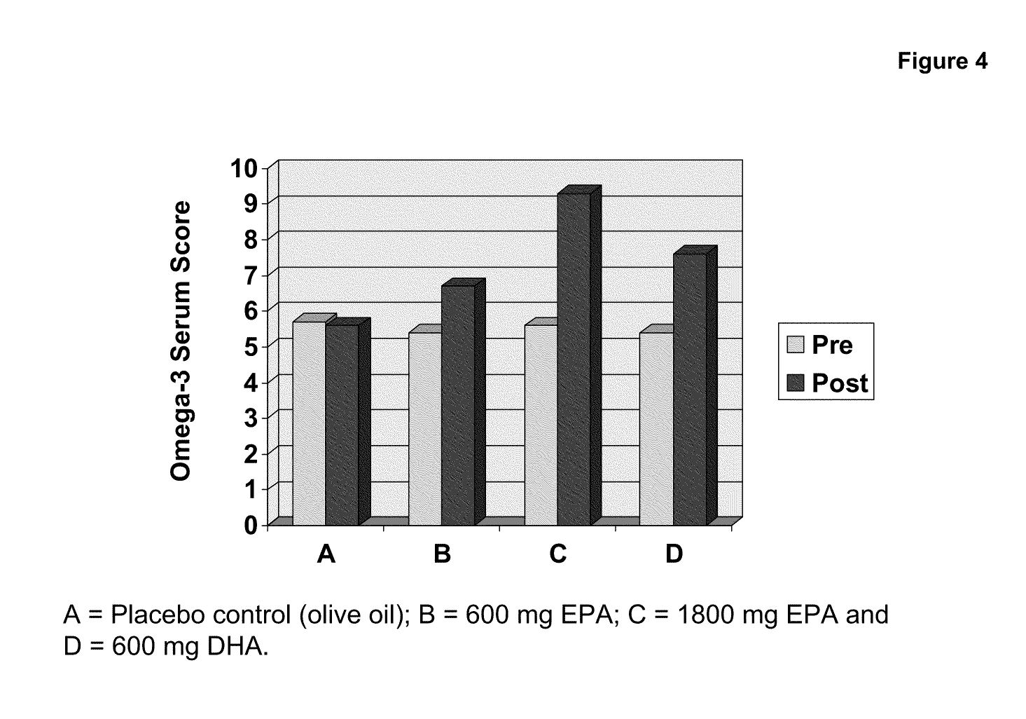

The EPA-rich oil, but not the DHA-rich oil, significantly raised the serum level of EPA (FIG. 1 and FIG. 2) and significantly decreased the serum ratio of ARA/EPA (FIG. 3) in a dose-dependent manner. There was no indication of metabolic conversion of EPA to DHA or retroconversion of DHA to EPA which is notable and relevant to subsequent discussions related to effects of EPA on Lp-PLA₂ (vidae infra). As expected, both the EPA and DHA-rich oils increased the Total Omega-3 Scores™ (FIG. 4).

### B2. Adverse Effects and Safety Testing

Of 121 subjects enrolled in the study, 110 completed the 6 week protocol. There were no major adverse effects, and non-completion was related to lack of compliance. Capsules were well tolerated. A fishy odor with belching was occasionally experienced by 15%, 28%, and 39%, respectively, in the active groups (EPA 600 mg, EPA 1800 mg, and DHA 600 mg), versus 8% in the olive oil group. No significant effects versus baseline values of any study intervention on the following parameters were noted: blood urea nitrogen, creatinine, calculated glomerular filtration rate, sodium, potassium, chloride, carbon dioxide, calcium, total protein, albumin, globulin, total bilirubin, alkaline phosphatase, liver transaminases AST and ALT, fasting glucose, complete blood count including hemoglobin, hematocrit, red blood cell count, platelet count, white blood cell count and a white blood cell count differential, prothrombin time, or thyroid function tests (T3, T4 and T3 uptake).

### B3. Effects on Body Weight, Blood Pressure, Glucose and Insulin Levels

No significant effects versus baseline values of any study intervention on body weight, body mass index [“BMI”], systolic blood pressure [“BP”], diastolic blood pressure [“BP”], pulse, fasting glucose or insulin levels were noted, except for the EPA 600 mg/day group where a modest, but significant 5.5% increase in diastolic blood pressure was observed (see Tables 3A, 3B, 3C and 3D).

### B4. Effects on Plasma Lipids, Apolipoproteins, and Inflammatory Markers

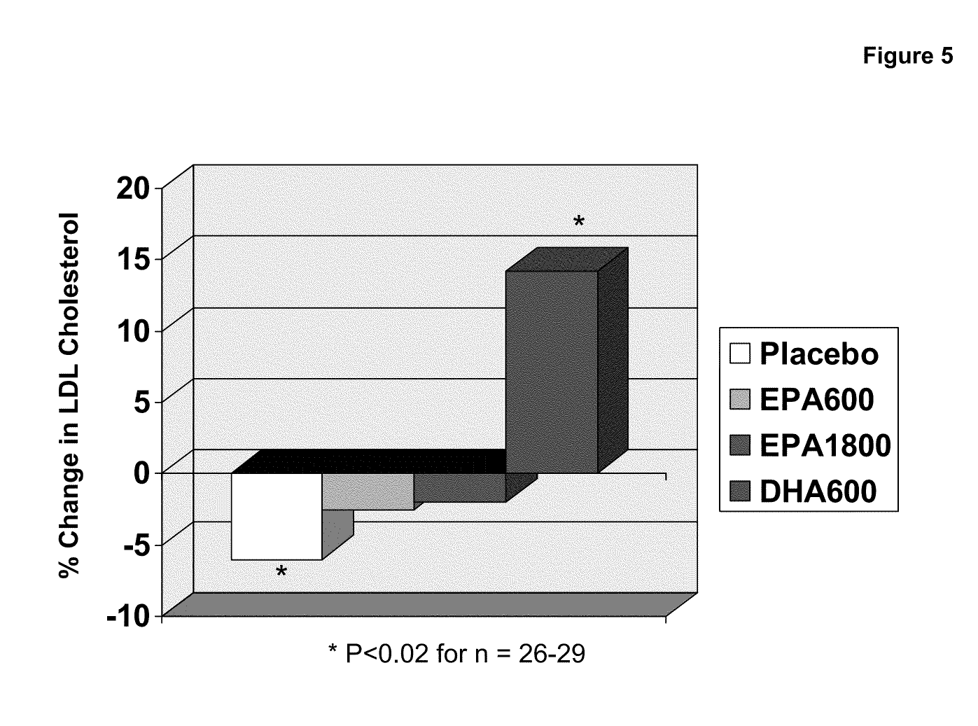

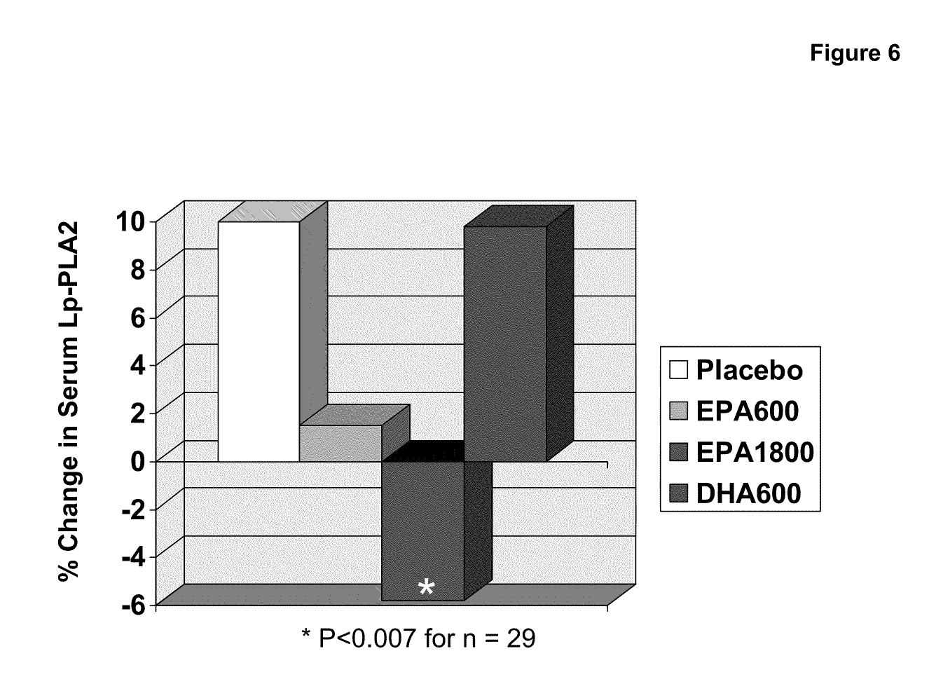

Data on changes in plasma lipids (total cholesterol, LDL cholesterol, HDL cholesterol, triglycerides, small dense LDL cholesterol (sdLDL), apolipoproteins (apoA-I, apoB, Lp(a)), insulin, and markers of inflammation (high sensitivity C reactive protein [“hsCRP”], IL-6, and Lp-PLA₂), and adhesion molecules soluble ICAM [“sICAM”] and VCAM are shown in Tables 4A, 4B, 4C and 4D and FIG. 4, FIG. 5 and FIG. 6. For those receiving olive oil, there was a significant 6.0% reduction in LDL cholesterol and a significant 7.1% increase in HDL cholesterol in the fasting state versus baseline values, with similar trends observed in the fed state. There was also a 10.0% increase in Lp-PLA₂ (p=0.053). For those receiving EPA 600 mg/day, a significant decrease of 7.3% (p=0.0087) versus baseline was noted for small dense LDL cholesterol. For those receiving EPA 1800 mg/day, a significant decrease of 8.8% (p=0.018) versus baseline was noted for small dense LDL cholesterol. For those receiving EPA 1800 mg/day, a significant decrease of 8.8% (p=0.018) versus baseline was noted for small dense LDL cholesterol, and a significant decrease of 5.8% (p=0.01) versus baseline was noted for Lp-PLA₂, with trends towards reductions in fasting triglyceride levels (−5.0%, p=0.08). For those receiving DHA 600 mg/day, significant increases were noted in fasting LDL cholesterol of 14.2% (p=0.02), and fed LDL cholesterol of 16.3% (p=0.001). Trends for increases in Lp-PLA₂ (+9.8%, p=0.06) and decreases in post-prandial triglycerides (−9.5%, p=0.051) were noted. No significant effects of any of these interventions on insulin, CRP and IL6 or adhesion molecules ICAM and VCAM or other cardiovascular risk factors were noted. Regression analysis of EPA versus Lp-PLA₂ was statistically significant; notably, this was not the case for DHA.

### C. Discussion of Results

The overall data discussed herein indicate that the beneficial effects of high dose EPA on CVD risk reduction could be related to decreases in Lp-PLA₂, a marker of inflammation in the arterial wall. The mechanisms whereby EPA causes this effect may well relate to an overall inhibition of the cellular immune response, as well as an inhibition of white blood cell recruitment into the artery wall. The overall cardioprotective effects of omega-3 fatty acids have been reviewed by Harris and colleagues (Harris W. S. et al., Atherosclerosis, 197:12-24 (2008)). Despite studies such as GISSI and JELIS, the focus of coronary heart disease risk reduction remains on LDL lowering (Executive Summary Of The 3^{rd} Report Of The National Cholesterol Education Program [“NCEP”] Expert Panel, J. Am. Med. Assoc., 285:2486-2497 (2001)). However, this focus may well change since many patients with heart disease still experience significant residual risk despite being on statin therapy. Fish oil supplementation has been shown to be beneficial for coronary heart disease risk reduction, but the roles of DHA and EPA appear to be different, with DHA being more effective in triglyceride lowering and arrhythmia prevention, while EPA may be more effective in decreasing the inflammatory response within the artery wall, thereby decreasing risk of atherosclerosis progression.

TABLE 2
Race and Gender Demographics

| | | | Olive | Olive | EPA | EPA | EPA | EPA | DHA | DHA |
|---|---|---|---|---|---|---|---|---|---|---|
| | All | All % | Oil | Oil % | 600 mg | 600 mg % | 1800 mg | 1800 mg % | 600 mg | 600 mg % |
| White | 77 | 70.0 | 19 | 73.1 | 20 | 74.1 | 18 | 62.1 | 20 | 71.4 |
| Black | 28 | 25.5 | 6 | 23.1 | 6 | 22.2 | 9 | 31.0 | 7 | 25.0 |
| Asian | 3 | 2.7 | 1 | 3.8 | 0 | 0.0 | 2 | 6.9 | 0 | 0.0 |
| Hispanic | 2 | 1.8 | 0 | 0.0 | 1 | 3.7 | 0 | 0.0 | 1 | 3.6 |
| Total | 110 | | 26 | | 27 | | 29 | | 28 | |
| Male | 74 | 67.3 | 18 | 69.2 | 18 | 66.7 | 19 | 65.5 | 19 | 67.9 |
| Female | 36 | 32.7 | 8 | 30.8 | 9 | 33.3 | 10 | 34.5 | 9 | 32.1 |
| Total | 110 | | 26 | | 27 | | 29 | | 28 | |

TABLE 3A
Heart Disease Risk Factors at Baseline and % Change at 6 Weeks (Group A)
Olive Oil Placebo (n = 26)

| Variable | Baseline | Final | 6-Week Change (%) | P-Value for Change |
|---|---|---|---|---|
| Weight (kg) | 85.9 ± 17.0 | 86.2 ± 16.9 | +0.4 ± 1.7 | 0.32 |
| BMI (kg/m²) | 27.7 ± 4.7 | 27.7 ± 4.7 | +0.1 ± 1.9 | 0.79 |
| Systolic BP (mm Hg) | 121.9 ± 13.0 | 120.8 ± 11.3 | −0.5 ± 7.8 | 0.57 |
| Diastolic BP (mm | 78.1 ± 8.5 | 78.7 ± 6.9 | +1.5 ± 10.8 | 0.74 |
| Pulse (beats/min) | 71.7 ± 8.3 | 73.2 ± 8.8 | +2.6 ± 11.6 | 0.37 |
| Insulin (μIU/L) | 13.2 ± 19.3 | 10.5 ± 9.8 | +1.1 ± 52.9 | 0.26 |
| Glucose (mg/dL) | 93.5 ± 15.8 | 92.4 ± 14.1 | −1.6 ± 10.6 | 0.60 |

Mean values and percentage changes at 6 weeks, with standard deviations

TABLE 3B
Heart Disease Risk Factors at Baseline and % Change at 6 Weeks (Group B)
EPA 600 mg (n = 27)

| Variable | Baseline | Final | 6-Week Change (%) | P-Value for Change |
|---|---|---|---|---|
| Weight (kg) | 80.4 ± 11.3 | 80.4 ± 10.9 | +0.2 ± 2.1 | 0.83 |
| BMI (kg/m²) | 27.4 ± 3.1 | 27.4 ± 3.1 | +0.2 ± 2.3 | 0.64 |
| Systolic BP (mm Hg) | 118.5 ± 14.7 | 121.1 ± 12.2 | +3.0 ± 10.4 | 0.27 |
| Diastolic BP (mm | 76.4 ± 8.9 | 80.0 ± 7.9 | +5.5 ± 11.5 | 0.039 |
| Pulse (beats/min) | 73.6 ± 11.0 | 74.7 ± 12.0 | +2.1 ± 13.6 | 0.56 |
| Insulin (μIU/L) | 10.3 ± 8.0 | 10.2 ± 7.9 | +17.4 ± 70.6 | 0.89 |
| Glucose (mg/dL) | 90.0 ± 11.0 | 92.1 ± 11.0 | +1.3 ± 14.0 | 0.47 |

Mean values and percentage changes at 6 weeks, with standard deviations

TABLE 3C
Heart Disease Risk Factors at Baseline and % Change at 6 Weeks (Group C)
EPA 1800 mg (n = 29)

| Variable | Baseline | Final | 6-Week Change (%) | P-Value for Change |
|---|---|---|---|---|
| Weight (kg) | 80.4 ± 18.0 | 81.6 ± 19.2 | +1.3 ± 4.6 | 0.19 |
| BMI (kg/m2) | 27.5 ± 4.6 | 27.9 ± 5. | +1.1 ± 5.1 | 0.24 |
| Systolic BP (mm Hg) | 119.3 ± 15.5 | 119.9 ± 13.4 | +1.0 ± 7.4 | 0.71 |
| Diastolic BP (mm | 76.6 ± 8.6 | 77.4 ± 9.0 | +1.4 ± 9.1 | 0.52 |
| Pulse (beats/min) | 69.3 ± 8.8 | 70.9 ± 8.5 | +2.7 ± 8.6 | 0.22 |
| Insulin (μIU/L) | 8.1 ± 6.3 | 7.2 ± 6.2 | +2.4 ± 57.6 | 0.25 |
| Glucose (mg/dL) | 91.7 ± 8.9 | 91.9 ± 7.7 | +0.01 ± 8.5 | 0.87 |

Mean values and percentage changes at 6 weeks, with standard deviations

TABLE 3D
Heart Disease Risk Factors at Baseline and % Change at 6 Weeks (Group D)
DHA 1800 mg (n = 28)

| Variable | Baseline | Final | 6-Week Change (%) | P-Value for Change |
|---|---|---|---|---|
| Weight (kg) | 80.6 ± 16.0 | 81.1 ± 15.7 | +0.8 ± 2.4 | 0.16 |
| BMI (kg/m2) | 27.0 ± 4.3 | 27.1 ± 4.0 | +0.3 ± 2.3 | 0.80 |
| Systolic BP (mm Hg) | 125.6 ± 15.8 | 126.1 ± 13.4 | +1.0 ± 7.7 | 0.79 |
| Diastolic BP (mm | 81.4 ± 11.4 | 80.8 ± 9.4 | +0.0 ± 8.7 | 0.71 |
| Pulse (beats/min) | 71.4 ± 13.1 | 72.1 ± 11.4 | +2.3 ± 14.9 | 0.76 |
| Insulin (μIU/L) | 8.9 ± 5.8 | 8.4 ± 4.1 | +26.7 ± 79.8 | 0.58 |
| Glucose (mg/dL) | 94.8 ± 14.5 | 96.4 ± 19.1 | +0.7 ± 9.5 | 0.42 |

Mean values and percentage changes at 6 weeks, with standard deviations

TABLE 4A
Serum Lipid and Lipoprotein Test Values at Baseline and % Change at 6 Weeks (Group A)
Olive Oil Placebo (n = 26)

| | | | | |
|---|---|---|---|---|
| Variables (mg/dl) | Baseline | Final | 6-Week Change (%) | P-Value for Change |

Fast

| | | | | |
|---|---|---|---|---|
| Total Cholesterol | 207.6 ± 42.3 | 206.4 ± 44.9 | −0.4 ± 8.1 | 0.74 |
| HDL C | 55.7 ± 18.4 | 59.7 ± 20.7 | +7.1 ± 15.3 | 0.028 |
| LDL C | 128.2 ± 34.4 | 120.6 ± 36.4 | −6.0 ± 11.4 | 0.012 |
| Triglyceride | 112.0 ± 54.5 | 123.1 ± 96.4 | +5.9 ± 42.5 | 0.35 |
| apoA-I | 166.3 ± 35.8 | 172.9 ± 36.9 | +4.5 ± 10.7 | 0.07 |
| sdLDL | 33.8 ± 13.8 | 31.4 ± 14.1 | −5.9 ± 29.3 | 0.24 |
| apoB | 95.3 ± 24.5 | 93.0 ± 26.4 | −2.4 ± 11.1 | 0.26 |
| hsCRP | 2.6 ± 4.8 | 2.1 ± 2.0 | +29.6 ± 68.1 | 0.55 |
| Lp(a) | 37.5 ± 47.1 | 34.0 ± 35.6 | +1.8 ± 35.9 | 0.29 |
| Lp-PLA₂ (ng/mL) | 168.5 ± 54.5 | 182.2 ± 58.3 | +10.0 ± 20.8 | 0.053 |
| Insulin | 13.2 ± 19.3 | 10.5 ± 9.8 | +1.1 ± 52.9 | 0.26 |
| sICAM (ng/mL) | 234.8 ± 91.8 | 231.8 ± 96.2 | −1.3 ± 10.6 | 0.59 |
| VCAM (ng/mL) | 674.4 ± 184.4 | 660.9 ± 158.4 | −1.1 ± 6.4 | 0.17 |
| IL-6 (pg/mL) | 1.6 ± 0.9 | 1.6 ± 0.8 | +11.7 ± 35.9 | 0.92 |
| Adiponectin (ng/mL) | 10030.1 ± 6812.9 | 12599.5 ± 9803.5 | 44.1 ± 164.6 | 0.081 |

Post Prandial

| | | | | |
|---|---|---|---|---|
| Total Cholesterol | 205.7 ± 39.0 | 204.7 ± 46.4 | −0.6 ± 11.0 | 0.84 |
| HDL C | 51.9 ± 16.4 | 53.6 ± 19.9 | +2.6 ± 17.8 | 0.39 |
| LDL C | 120.8 ± 32.6 | 114.6 ± 36.7 | −5.5 ± 15.8 | 0.09 |
| Triglyceride | 197.5 ± 103.8 | 213.4 ± 127.5 | +12.9 ± 47.3 | 0.34 |

Mean values and percentage changes at 6 weeks, with standard deviations

TABLE 4B
Serum Lipid and Lipoprotein Test Values at Baseline and % Change at 6 Weeks (Group B)
EPA 600 mg (n = 27)

| | | | | |
|---|---|---|---|---|
| Variables (mg/dl) | Baseline | Final | 6-Week Change (%) | P-Value for Change |

Fast

| | | | | |
|---|---|---|---|---|
| Total Cholesterol | 202.9 ± 45.3 | 199.2 ± 48.7 | −1.8 ± 9.5 | 0.35 |
| HDL C | 57.1 ± 14.1 | 57.9 ± 18.0 | +0.4 ± 13.7 | 0.64 |
| LDL C | 122.4 ± 37.1 | 118.1 ± 35.8 | −2.5 ± 11.7 | 0.16 |
| Triglyceride | 116.1 ± 56.2 | 107.0 ± 43.2 | −1.3 ± 31.6 | 0.25 |
| sdLDL | 32.9 ± 14.7 | 29.5 ± 12.0 | −7.3 ± 18.2 | 0.0087 |
| apoA-I | 175.4 ± 25.3 | 173.6 ± 36.3 | −1.3 ± 12.4 | 0.69 |
| apoB | 93.1 ± 23.8 | 90.1 ± 22.8 | −2.3 ± 10.9 | 0.17 |
| hsCRP | 2.3 ± 2.5 | 3.3 ± 4.9 | +115.6 ± 508.4 | 0.19 |
| Lp(a) | 31.7 ± 32.3 | 32.8 ± 32.4 | +18.3 ± 84.8 | 0.72 |
| Lp-PLA₂(ng/mL) | 170.0 ± 50.7 | 168.7 ± 44.9 | +1.5 ± 16.1 | 0.82 |
| Insulin | 10.3 ± 8.0 | 10.2 ± 7.9 | +17.4 ± 70.6 | 0.89 |
| sICAM (ng/mL) | 226.5 ± 50.6 | 232.4 ± 62.7 | +3.2 ± 19.0 | 0.46 |
| VCAM (ng/mL) | 718.0 ± 198.9 | 722.8 ± 178.0 | +2.2 ± 12.6 | 0.75 |
| IL-6 (pg/mL) | 1.9 ± 1.9 | 2.0 ± 1.8 | +35.3 ± 126.1 | 0.53 |
| Adiponectin (ng/mL) | 9341.1 ± 7155.8 | 9501.4 ± 5894.3 | 11.3 ± 31.4 | 0.83 |

Post Prandial

| | | | | |
|---|---|---|---|---|
| Total Cholesterol | 195.0 ± 46.8 | 193.3 ± 44.7 | −0.3 ± 8.6 | 0.60 |
| HDL C | 51.3 ± 15.7 | 53.1 ± 16.4 | +4.8 ± 20.7 | 0.28 |
| LDL C | 111.4 ± 35.3 | 108.2 ± 32.2 | −1.4 ± 12.9 | 0.26 |
| Triglyceride | 206.5 ± 107.3 | 184.9 ± 98.5 | −3.1 ± 36.9 | 0.17 |

Mean values and percentage changes at 6 weeks, with standard deviations

TABLE 4C
Serum Lipid and Lipoprotein Test Values at Baseline and % Change at 6 Weeks (Group C)
EPA 1800 mg (n = 29)

| | | | | |
|---|---|---|---|---|
| Variables (mg/dl) | Baseline | Final | 6-Week Change (%) | P-Value for Change |

Fast

| | | | | |
|---|---|---|---|---|
| Total Cholesterol | 206.9 ± 39.4 | 201.1 ± 35.3 | −1.7 ± 11.2 | 0.21 |
| HDL C | 58.5 ± 13.8 | 59.9 ± 15.7 | +2.5 ± 12.7 | 0.34 |
| LDL C | 124.8 ± 29.0 | 120.6 ± 26.8 | −2.0 ± 13.7 | 0.20 |
| Triglyceride | 114.3 ± 92.9 | 102.8 ± 82.8 | −5.0 ± 28.6 | 0.08 |
| sdLDL | 34.9 ± 12.9 | 30.6 ± 9.4 | −8.8 ± 19.5 | 0.018 |
| apoA-I | 173.5 ± 31.0 | 173.1 ± 31.3 | +0.4 ± 10.8 | 0.91 |
| apoB | 94.5 ± 21.7 | 91.7 ± 19.1 | −1.6 ± 12.2 | 0.21 |
| hsCRP | 2.6 ± 3.1 | 2.7 ± 4.7 | +26.0 ± 113.0 | 0.95 |
| Lp(a) | 33.4 ± 25.3 | 32.2 ± 23.6 | +5.1 ± 26.1 | 0.65 |
| Lp-PLA₂(ng/mL) | 145.5 ± 29.4 | 135.3 ± 24.8 | −5.8 ± 12.9 | 0.01 |
| Insulin | 8.1 ± 6.3 | 7.2 ± 6.2 | +2.4 ± 57.6 | 0.25 |
| sICAM (ng/mL) | 214.4 ± 49.6 | 206.9 ± 50.5 | −2.6 ± 17.1 | 0.24 |
| VCAM (ng/mL) | 614.1 ± 172.9 | 607.5 ± 125.7 | +1.0 ± 13.5 | 0.75 |
| IL-6 (pg/mL) | 2.5 ± 5.2 | 1.3 ± 0.7 | +3.1 ± 50.1 | 0.22 |
| Adiponectin (ng/mL) | 12328.7 ± 18667.5 | 17578.6 ± 45606.9 | 16.4 ± 82.6 | 0.49 |

Post Prandial

| | | | | |
|---|---|---|---|---|
| Total Cholesterol | 202.3 ± 39.9 | 197.3 ± 33.8 | −1.4 ± 9.9 | 0.21 |
| HDL C | 53.4 ± 13.4 | 55.3 ± 15.3 | +3.4 ± 11.5 | 0.09 |
| LDL C | 116.2 ± 27.9 | 111.5 ± 25.2 | −2.7 ± 13.6 | 0.14 |
| Triglyceride | 209.3 ± 167.3 | 193.5 ± 184.2 | −3.8 ± 40.2 | 0.15 |

Mean values and percentage changes at 6 weeks, with standard deviations

TABLE 4D
Serum Lipid and Lipoprotein Test Values at Baseline and % Change at 6 Weeks (Group D)
DHA 600 mg (n = 28)

| | | | | |
|---|---|---|---|---|
| Variables (mg/dl) | Baseline | Final | 6-Week Change (%) | P-Value for Change |

Fast

| | | | | |
|---|---|---|---|---|
| Total Cholesterol | 210.9 ± 38.7 | 216.1 ± 44.1 | +2.9 ± 12.0 | 0.26 |
| HDL C | 62.6 ± 26.4 | 62.0 ± 23.7 | +1.3 ± 14.7 | 0.75 |
| LDL C | 119.1 ± 36.1 | 130.3 ± 37.2 | +14.2 ± 36.6 | 0.02 |
| Triglyceride | 144.0 ± 192.9 | 104.4 ± 58.0 | −5.8 ± 30.4 | 0.22 |
| sdLDL | 33.4 ± 16.3 | 31.1 ± 13.3 | −2.2 ± 24.7 | 0.20 |
| apoA-I | 182.1 ± 44.4 | 177.5 ± 43.0 | −2.0 ± 10.5 | 0.18 |
| apoB | 93.5 ± 27.7 | 97.1 ± 25.7 | +6.0 ± 15.6 | 0.23 |
| hsCRP | 2.5 ± 3.4 | 3.4 ± 4.5 | +27.1 ± 82.3 | 0.16 |
| Lp(a) | 37.4 ± 35.9 | 40.0 ± 40.1 | +27.7 ± 156.3 | 0.23 |
| Lp-PLA₂(ng/mL) | 167.5 ± 40.6 | 180.0 ± 40.9 | +9.8 ± 21.2 | 0.06 |
| Insulin | 8.9 ± 5.8 | 8.4 ± 4.1 | +26.7 ± 79.8 | 0.58 |
| sICAM (ng/mL) | 241.3 ± 115.4 | 240.2 ± 114.1 | +0.7 ± 18.0 | 0.89 |
| VCAM (ng/mL) | 672.6 ± 186.0 | 673.0 ± 197.4 | +0.2 ± 11.0 | 0.97 |
| IL-6 (pg/mL) | 1.7 ± 1.0 | 1.6 ± 0.9 | +5.7 ± 50.3 | 0.69 |
| Adiponectin (ng/mL) | 15317.1 ± 32525.4 | 11380.0 ± 14772.6 | −6.7 ± 19.8 | 0.29 |

Post Prandial

| | | | | |
|---|---|---|---|---|
| Total Cholesterol | 200.3 ± 39.6 | 211.1 ± 38.6 | +6.2 ± 10.8 | 0.011 |
| HDL C | 57.4 ± 23.8 | 57.5 ± 22.7 | +1.2 ± 10.9 | 0.93 |
| LDL C | 107.8 ± 32.8 | 121.9 ± 32.9 | +16.3 ± 25.3 | 0.001 |
| Triglyceride | 210.9 ± 167.1 | 169.8 ± 89.2 | −9.5 ± 24.4 | 0.051 |

Mean values and percentage changes at 6 weeks, with standard deviations

TABLE 5
Capsule Fatty Acid Compositions (mg fatty acid/g of oil *)

| | C18:1n9 | C20:5 n3 | Total | Total Mono- | Total Poly- | Total |
|---|---|---|---|---|---|---|
| | (Oleic Acid) | (EPA) | Saturates | unsaturates | unsaturates | Omega 3 |
| Olive Oil | 659.0 | 0.0 | 150.3 | 680.2 | 97.9 | 5.6 |
| 100 mg EPA oil | 484.3 | 103.8 | 125.8 | 506.3 | 263.9 | 128.9 |
| 300 mg EPA oil | 143.1 | 314.8 | 76.1 | 160.7 | 600.1 | 379.6 |

* Fatty acid composition quantified as mg FA/g of oil can be converted to the % FA in the oil by dividing mg FA/g of oil by a factor of 10. Thus, for example, 314.8 mg EPA/g of oil is equivalent to 31.48% EPA in the oil.

1. A method for maintaining or lowering Lp-PLA₂ levels in a normal subject which comprises administering an effective amount of EPA.

2-25. (canceled)

26. A method for maintaining or lowering lipoprotein-associated phospholipase A₂ (Lp-PLA₂) levels in a human subject, wherein the method comprises:
(a) identifying a human subject that (i) is not taking a dyslipidemic agent and (ii) has a serum Lp-PLA₂ level that is below 200 ng/mL,
(b) administering to the subject an effective amount of eicosapentaenoic acid (EPA) that is substantially free of docosahexaenoic acid (DHA), and
(c) optionally screening the subject for a maintained or lowered serum Lp-PLA₂ level;
wherein the administration of EPA in step (b) maintains or lowers the level of serum Lp-PLA₂ in the subject.

27. The method of claim 26, wherein the EPA is in a triglyceride form in an oil that is low in saturated fatty acids.

28. The method of claim 26, further comprising:
(i) determining the level of low density lipoprotein (LDL) cholesterol in the serum of the subject in step (a), and
(ii) optionally determining the level of LDL cholesterol in the serum of the subject in step (c);
wherein the level of LDL cholesterol in the serum of the subject in step (c) is not increased compared to the level of LDL cholesterol in the serum of the subject in step (a).

29. The method of claim 28, further comprising:
(iii) determining the level of serum EPA of the subject in step (a);
wherein the subject in step (a) has a low serum EPA level.

30. The method of claim 26, wherein the effective amount of EPA comprises less than about 0.1% by weight of DHA.

31. The method of claim 27, wherein the oil comprises at least about 30.0% by weight EPA in the total fatty acids of the oil.

32. The method of claim 31, wherein the oil comprises less than about 15.0% by weight saturated fatty acids in the total fatty acids of the oil.

33. The method of claim 26, wherein the effective amount of EPA is at least about 500 mg/day.

34. The method of claim 33, wherein the effective amount of EPA is at least about 1200 mg/day.

35. The method of claim 33, wherein said administering step (b) is carried out daily over a period of six weeks.

36. The method of claim 26, including the step of screening the subject for a maintained or lowered serum Lp-PLA₂ level.

37. The method of claim 27, wherein the oil comprises less than 0.5% gamma-linolenic acid in the total fatty acids of the oil.
